(19) United States
(12) Patent Application Publication
(10) Pub. No.: US 2008/0242599 A1
(43) Pub. Date: Oct. 2, 2008

(54) Compositions and Methods for Increasing Osteoblast Cell Differentiation and Bone Generation

(76) Inventors: Majd Zayzafoon, Birmingham, AL (US); Jay M. McDonald, Birmingham, AL (US); Hyeonju Yeo, Birmingham, AL (US)

Correspondence Address: SUTHERLAND ASBILL & BRENNAN LLP, 999 PEACHTREE STREET, N.E., ATLANTA, GA, 30309, US

(21) Appl. No.: 11/913,643

(22) PCT Filed: May 3, 2006
(86) PCT No.: PCT/US06/17104

§ 371 Date: Jun. 5, 2008

Related U.S. Application Data
(60) Provisional application No. 60/677,035, filed on May 3, 2005.
(60) Provisional application No. 60/740,931, filed on Nov. 30, 2005.

(51) Int. Cl. A61K 38/13; C12Q 1/02; A61K 31/436; A61P 19/00; C12N 5/06

(52) U.S. Cl. 514/11; 435/29; 435/377; 435/325; 514/291

(57) ABSTRACT

The present invention relates to the discovery of a novel pathway for the induction of osteoblast cellular differentiation and bone generation. Specifically, the present invention envisions a novel screening tool for the determination of compounds capable of promoting osteoblast cellular differentiation that could be used in the treatment of various bone-loss or bone density decreasing disorders. The method of screening of the present invention enables one to determine whether a compound affects certain pathways that promote via direct induction, or the downregulation of inhibiting activity, the differentiation of progenitor cells into osteoblasts. This promotion of osteoblasts could provide for treatments for bone-loss or bone density disorders. The invention further encompasses methods of increasing bone density using the compounds, the compounds, and pharmaceutical compositions comprising the compounds.

### 1 ACKNOWLEDGMENT OF FEDERAL RESEARCH SUPPORT

This invention was made, at least in part, with funding from the National Institutes of Health (Award Number 1R01-AR050235). Accordingly, the United States Government has certain rights in this invention.

### 1 FIELD OF THE INVENTION

The present invention relates to the discovery of a novel pathway for the induction of osteoblast cellular differentiation and bone generation. Specifically, the present invention envisions a novel screening tool for the determination of compounds capable of promoting osteoblast cellular differentiation that could be used in the treatment of various bone-loss or bone density decreasing disorders.

### 1 BACKGROUND

Bone loss and osteoporosis are major public health problems in the elderly. With people in the United States living longer than before, the number of people that will develop age-related bone loss and osteoporosis is expected to rise drastically in the coming decades. Osteoporosis not only presents problems in and of itself, but with joint replacement and musculoskeletal disorders requiring manipulation and repair of bone or boney tissue, depleted bone mass presents additional problems to an aging population.

Bone is comprised of several different cell types. Osteoblasts lay down new bone from the minerals present in the extracellular milieu around the cells. Osteoclasts remove old bone releasing the minerals compiled within bone back into the extracellular matrix. This balance between adequate new bone being deposited and old bone being removed is what gives bone its extremely beneficial properties. Osteoblasts originate from mesenchymal stem cells while osteoclasts originate from hematopoietic stem cells.

Osteoblast differentiation is a crucial aspect of bone formation and remodeling. Osteoporosis is one disorder that reflects a flaw in this delicate balance.

The process of new bone formation involves the recruitment of osteoprogenitor cells that, with the appropriate stimulation, undergo proliferation and differentiate into preosteoblasts and then into mature osteoblasts to synthesize inorganic matrix into mineralized bone.

It has been documented that bone disease, in the form of osteopenia and osteoporosis, is a serious complication of organ transplantation in humans (Shane et al., (1997) J Clin Endocrinol Metab 82:1497-1506; Shane et al., (1993) Am J Med 94:257-264; Katz & Epstein, (1992) J Bone Miner Res 7:123-126). This phenomenon is not only due to the underlying disease and the organ transplantation but also is a side effect of the drugs that are administered pre- and post- transplantation, including cyclosporine (CsA) (Katz & Epstein, (1992) J Bone Miner Res 7:123-126; Mazanec & Grisanti, (1989) Cleve Clin J Med 56:297-303; Lukert & Raisz, (1994) Rheum Dis Clin North Am 20:629-650; Rich et al., (1992) J Heart Lung Transplant 11:950-958). The co-administration of CsA with other agents in humans makes it difficult to determine specific effects of CsA monotherapy on bone. In animals, the effects of CsA treatment on bone have been contradictory. It has been reported that the administration of CsA to rats produces severe osteopenia with either a significant increase or decrease in bone remodeling and turnover (Movsowitz et al., (1988) Endocrinology 123:2571-2577; Movsowitz et al., (1989) J Bone Miner Res 4:393-398; Igarashi et al., (2004) Bone 35:47-56; Fu et al., (2001) Arch Oral Biol 46:1105-1110). It is unclear why CsA treatment could produce a high turnover bone loss (increase in osteoblasts and osteoclasts) in one animal model and low turnover bone loss in another (decrease in osteoblasts and osteoclasts). Due to the osteopenic effects of CsA, almost all studies have been conducted to examine the effects of CsA on osteoclasts and bone resorption. Its role in osteoblast differentiation and bone formation is not known.

Recently, the majority of osteoporosis treatment has been with anti-resorptive agents such as estrogens, biphosphonates, and calcitonin (Black et al., (1996) Lancet 348:1535-1541; Chesnut et al, (2000) Am J Med 109:267-276; Ettinger et al., (1999) JAMA 282:637-645). The major function of these therapeutic agents is to decrease bone resorption, as opposed to increase new bone generation. Recombinant human parathyroid hormone (rhPTH) has been used as an anabolic therapeutic treatment specifically targeting osteoblasts (Rosen, (2004) Trends Endocrinol Metab 15:229-233). With notable complications and limitations, finding a new anabolic agent that will promote osteoblast function is important.

### SUMMARY OF THE INVENTION

The present invention relates to a method for screening for a compound that is capable of inducing an increase in osteoblast differentiation. The present invention encompasses a method of identifying a compound capable of increasing osteoblast cell differentiation, comprising: a) providing a bone precursor cell, b) contacting the bone precursor cell with a test compound, and c) determining whether a decrease in nuclear translocation of a member of the NFAT superfamily occurs in the cell contacted with the compound, said decrease being an indication that the compound increases osteoblast cell differentiation.

The invention further encompasses a method of identifying a compound capable of increasing osteoblast cell differentiation, comprising: a) providing a bone precursor cell expressing a member of the NFAT superfamily, b) contacting the bone precursor cell with a test compound, and c) determining whether an increase of Runx2 expression occurs in the cell contacted with the compound, said increase being an indication that the compound increases osteoblast cell differentiation. In a further embodiment, contact with the test compound results in a decrease in dephosphorylation of the member of the NFAT superfamily.

The invention also encompasses a method of identifying a compound that decreases the dephosphorylation of a member of the NFAT superfamily, comprising: a) providing a bone precursor cell expressing a member of the NFAT superfamily, b) contacting the bone precursor cell with a test compound, and c) determining whether dephosphorylation of the member of the NFAT superfamily is decreased in the presence of the test compound, a decrease in said dephosphorylation being an indication that the test compound inhibits the dephosphorylation of the member of the NFAT superfamily. In one embodiment, contacting the bone precursor cell with the compound increases osteoblast cell differentiation.

It is preferred that the member of the NFAT superfamily is selected from the group consisting of NFAT_{c1}, NFAT_{c2}, NFAT_{c3}, NFAT_{c4}, and NFAT₅. In a further embodiment, the member of the NFAT superfamily is selected from the group consisting NFAT_{c}1 and NFAT_{c3}. In yet a further embodiment, the member of the NFAT superfamily is NFAT_{c1}. In one embodiment, the bone precursor cell expresses calcineurin, and the test compound decreases the binding of calcineurin to a member of the NFAT superfamily.

Also encompassed is a method of identifying a compound that inhibits the activity of calcineurin, comprising a) contacting calcineurin with a substrate for calcineurin and a test compound, and b) determining whether dephosphorylation of the substrate is decreased in the presence of the test compound, said decrease in dephosphorylation being an indication that the compound inhibits the activity of calcineurin. In one embodiment, calcineurin and the substrate for calcineurin are expressed in a bone precursor cell, and the bone precursor cell is contacted with the test compound. In one embodiment, contacting the bone precursor cell with the compound increases osteoblast cell differentiation.

The invention is also directed to a process for making a compound that increases osteoblast cell differentiation, comprising: carrying out any of the methods described herein to identify a compound that increases osteoblast cell differentiation, and manufacturing the compound.

The invention further encompasses a method of differentiating a bone precursor cell population comprising a) providing a bone precursor cell population, and b) contacting the bone precursor cell population with an effective amount of an inactivator of the calcineurin signaling pathway wherein differentiation of the bone precursor cell population to osteoblasts is increased in comparison to a bone precursor cell population that is not contacted with an effective dose of the inactivator of the calcineurin signaling pathway. In one embodiment, the bone precursor cell population is contacted with the inactivator for greater than approximately 12 hours, greater than approximately 24 hours, greater than approximately 48 hours, or greater than approximately 72 hours.

The invention also contemplates a composition comprising a homogenous population of differentiated cells, wherein the differentiated cells are differentiated from bone precursor cells in an in vitro culture, and wherein greater than approximately 25% of the population express Runx2, Fra-2, and alkaline phosphatase. In one embodiment, the differentiated cells also express at least one of osteocalcin, β-catenin, CCAAT/enhancer binding protein (C/EBP), and ATF4. In certain embodiments, greater than approximately 25%, greater than approximately 50%, greater than approximately 60%, greater than approximately 70%, greater than approximately 75%, greater than approximately 80%, greater than approximately 85%, greater than approximately 90%, or greater than approximately 95% of the differentiated cell population comprises osteoblasts. In other embodiments, the cells have been differentiated by contact with an effective amount of an inactivator of the calcineurin signaling pathway. In one embodiment, the inactivator is cyclosporine. In another embodiment, the inactivator is FK506.

The present invention further encompasses a method of improving bone mass in an individual in need thereof, comprising administering to the individual a therapeutically effective amount of a compound that decreases nuclear translocation of a member of the NFAT superfamily in a bone precursor cell. In a further embodiment, the compound decreases dephosphorylation of a member of the NFAT superfamily in a bone precursor cell. In another embodiment, the compound inhibits the activity of calcineurin in a bone precursor cell. In one embodiment, the activity of calcineurin is determined by examining dephosphorylation of a calcineurin substrate, wherein a decrease in dephosphorylation is an indication that the compound inhibits the activity of calcineurin.

The invention also encompasses pharmaceutical compositions capable of improving bone mass, comprising a therapeutically effective amount of a compound that decreases nuclear translocation of a member of the nuclear factor of activated T-cells (NFAT) superfamily in a bone precursor cell, whereby administration of the composition causes an improvement in bone mass in vivo. In another embodiment, the compound decreases the dephosphorylation of a member of the nuclear factor of activated T-cells (NFAT) superfamily in a bone precursor cell. In another embodiment, the compound inhibits the activity of calcineurin in a bone precursor cell. In one embodiment, the activity of calcineurin is determined by examining dephosphorylation of a calcineurin substrate, wherein a decrease in dephosphorylation is an indication that the compound inhibits the activity of calcineurin.

### BRIEF DESCRIPTION OF THE FIGURES

FIG. 1 shows a photograph of the results of an immunoblot developed using antibodies directed against total NFATc1, c2, and c3.

FIG. 2 shows a graph of the measurement of NFAT-luciferase activity within cells exposed to no CsA, 100 nM CsA, 500 nM CsA, and 1 μM CsA.

FIG. 3a shows a photograph of the results of staining of MC3T3-E1 cells for alkaline phosphatase activity and mineralization by von Kossa in cell cultures (14 days (top panel) or 21 days (lower panel)) treated with CsA every 2 days starting on day 4.

FIG. 3b shows a photograph of the results of staining of primary mouse calvarial osteoblasts for alkaline phosphatase activity and mineralization by von Kossa in cell cultures (14 days (top panel) or 21 days (lower panel)) treated with CsA every 2 days starting on day 4.

FIG. 3c shows a graph of the results of staining for FITC-conjugated annexin V and PI for detection of apoptotic cell death in cell cultures treated with CsA for 24 hours.

FIG. 3d shows a photograph of the results of staining for alkaline phosphatase activity in cell cultures treated with FK506.

FIG. 4a shows a graph of the measurement of the relative alkaline phosphatase expression in exposed cells versus concentration of CsA dosage.

FIG. 4b shows a graph of the measurement of the relative osteocalcin expression in exposed cells versus concentration of CsA dosage.

FIG. 5a shows a photograph of the results of staining for osteoblasts indicating an increase in cell differentiation.

FIG. 5b shows a photograph of the results of staining for mineralized bone indicating an increase in volume compared to total volume.

FIG. 6a shows a graph indicating the results of RT-PCR for fra-2.

FIG. 6b shows the results of immunoblots using antibodies directed against Fra-2 and total SP-1.

FIG. 6c shows the results of immunochemistry for Fra-2 after treatment with CsA.

FIG. 7a shows the results of immunoblots with antibodies against NFATc1.

FIG. 7b shows the results of immunoblots with antibodies against Fra-2.

FIG. 7c shows a photograph of the results of staining for alkaline phosphatase activity and mineralization by von Kossa in cell cultures transfected with GFP-empty vector (MSCV-GFP) or the constitutively-active NFATc1 (MSCV-NFATc1-GFP) retrovirus, and cultured for 10 days.

FIG. 7d shows an immunoblot of nuclear protein extracts from cells transfected with GFP-empty vector (MSCV-GFP) or the constitutively-active NFATc1 (MSCV-NFATc1-GFP) retrovirus, developed with antibodies against NFATc1, Fra-2 and SP-1.

FIG. 8 shows the results of immunoblots with antibodies directed against NFATc1 and β-catenin.

FIG. 9 shows a photograph of a histological analysis of bone in control mice and calcineurin conditional knockout mice.

### DETAILED DESCRIPTION OF THE INVENTION

The present invention may be understood more readily by reference to the following detailed description of the preferred embodiments of the invention and the Examples included herein. However, before the present compositions and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art.

The present invention relates to a method for screening for a compound that is capable of inducing an increase in osteoblast differentiation.

The present invention encompasses a method of identifying a compound capable of increasing osteoblast cell differentiation, comprising: a) providing a bone precursor cell, b) contacting the bone precursor cell with a test compound, and c) determining whether a decrease in nuclear translocation of a member of the NFAT superfamily occurs in the cell contacted with the compound, said decrease being an indication that the compound increases osteoblast cell differentiation.

The invention further encompasses a method of identifying a compound capable of increasing osteoblast cell differentiation, comprising: a) providing a bone precursor cell expressing a member of the NFAT superfamily, b) contacting the bone precursor cell with a test compound, and c) determining whether an increase of Runx2 expression occurs in the cell contacted with the compound, said increase being an indication that the compound increases osteoblast cell differentiation. In a further embodiment, contact with the test compound results in a decrease in dephosphorylation of the member of the NFAT superfamily.

The invention also encompasses a method of identifying a compound that decreases the dephosphorylation of a member of the NFAT superfamily, comprising: a) providing a bone precursor cell expressing a member of the NFAT superfamily, b) contacting the bone precursor cell with a test compound, and c) determining whether dephosphorylation of the member of the NFAT superfamily is decreased in the presence of the test compound, a decrease in said dephosphorylation being an indication that the test compound inhibits the dephosphorylation of the member of the NFAT superfamily. In one embodiment, contacting the bone precursor cell with the compound increases osteoblast cell differentiation.

Also encompassed is a method of identifying a compound that inhibits the activity of calcineurin, comprising a) contacting calcineurin with a substrate for calcineurin and a test compound, and b) determining whether dephosphorylation of the substrate is decreased in the presence of the test compound, said decrease in dephosphorylation being an indication that the compound inhibits the activity of calcineurin. In one embodiment, calcineurin and the substrate for calcineurin are expressed in a bone precursor cell, and the bone precursor cell is contacted with the test compound. In one embodiment, contacting the bone precursor cell with the compound increases osteoblast cell differentiation.

The invention is also directed to a process for making a compound that increases osteoblast cell differentiation, comprising: carrying out any of the methods described herein to identify a compound that increases osteoblast cell differentiation, and manufacturing the compound.

The invention further encompasses a method of differentiating a bone precursor cell population comprising a) providing a bone precursor cell population, and b) contacting the bone precursor cell population with an effective amount of an inactivator of the calcineurin signaling pathway wherein differentiation of the bone precursor cell population to osteoblasts is increased in comparison to a bone precursor cell population that is not contacted with an effective dose of the inactivator of the calcineurin signaling pathway. In one embodiment, the bone precursor cell population is contacted with the inactivator for greater than approximately 12 hours, greater than approximately 24 hours, greater than approximately 48 hours, or greater than approximately 72 hours.

The invention also contemplates a composition comprising a homogenous population of differentiated cells, wherein the differentiated cells are differentiated from bone precursor cells in an in vitro culture, and wherein greater than approximately 25% of the population express Runx2, Fra-2, and alkaline phosphatase. In one embodiment, the differentiated cells also express at least one of osteocalcin, β-catenin, CCAAT/enhancer binding protein (C/EBP), and ATF4. In certain embodiments, greater than approximately 25%, greater than approximately 50%, greater than approximately 60%, greater than approximately 70%, greater than approximately 75%, greater than approximately 80%, greater than approximately 85%, greater than approximately 90%, or greater than approximately 95% of the differentiated cell population comprises osteoblasts. In other embodiments, the cells have been differentiated by contact with an effective amount of an inactivator of the calcineurin signaling pathway. In one embodiment, the inactivator is cyclosporine, particularly low dosages of cyclosporine as disclosed herein. In another embodiment, the inactivator is FK506. Additional therapeutic inactivators, including those with structural homology to those identified herein, can be routinely identified and used in view of the present disclosure.

The present invention further encompasses a method of improving bone mass in an individual in need thereof, comprising administering to the individual a therapeutically effective amount of a compound that decreases nuclear translocation of a member of the NFAT superfamily in a bone precursor cell. In a further embodiment, the compound decreases dephosphorylation of a member of the NFAT superfamily in a bone precursor cell. In another embodiment, the compound inhibits the activity of calcineurin in a bone precursor cell. In one embodiment, the activity of calcineurin is determined by examining dephosphorylation of a calcineurin substrate, wherein a decrease in dephosphorylation is an indication that the compound inhibits the activity of calcineurin.

The invention also encompasses pharmaceutical compositions capable of improving bone mass, comprising a therapeutically effective amount of a compound that decreases nuclear translocation of a member of the nuclear factor of activated T-cells (NFAT) superfamily in a bone precursor cell, whereby administration of the composition causes an improvement in bone mass in vivo. In another embodiment, the compound decreases the dephosphorylation of a member of the nuclear factor of activated T-cells (NFAT) superfamily in a bone precursor cell. In another embodiment, the compound inhibits the activity of calcineurin in a bone precursor cell. In one embodiment, the activity of calcineurin is determined by examining dephosphorylation of a calcineurin substrate, wherein a decrease in dephosphorylation is an indication that the compound inhibits the activity of calcineurin.

It is preferred that the member of the NFAT superfamily is selected from the group consisting of NFATc1, NFATc2, NFATc3, NFATc4, and NFAT5. In a further embodiment, the member of the NFAT superfamily is selected from the group consisting NFATc1 and NFATc3. In yet a further embodiment, the member of the NFAT superfamily is NFATc1. In one embodiment, the bone precursor cell expresses calcineurin, and the test compound decreases the binding of calcineurin to a member of the NFAT superfamily.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

Ca²⁺ is a ubiquitous intracellular messenger responsible for controlling numerous cellular processes including fertilization, mitosis, neuronal transmission, contraction and relaxation of muscles, gene transcription, and cell death. At rest, the cytoplasmic Ca²⁺ concentration ([Ca²⁺]) is approximately 100 nM. This level can rise to 500-1000 nM upon activation. The elevation of [Ca²⁺] regulates cellular processes depending on the speed, amplitude, and spatio-temporal diversity of the increased [Ca²⁺]. Many of the cellular effects of Ca²⁺ are mediated by the Ca²⁺ binding protein, calmodulin (CaM). CaM is one of the most abundant, ubiquitous, and completely conserved proteins among vertebrates.

Structurally, CaM is a dumbbell shape formed by two globular domains at its C- and N-termini connected by a flexible helical linker region. Both globular ends contain a pair of Ca²⁺ binding motifs, and the binding of Ca²⁺ to CaM exposes a hydrophobic surface that is responsible for binding to various target proteins. The function of CaM is not confined to its Ca²⁺-bound form, as Ca²⁺-free CaM can also recognize different target proteins. More than 30 CaM-binding proteins have been identified, including enzymes such as kinases, phosphatases, and nitric-oxide synthase, as well as receptors, ion channels, G-proteins, and transcription factors.

The phosphatase, calcineurin, is of particular importance for the differentiation of osteoblasts because of its relation to NFAT. CaM not only mediates the effects of changes in [Ca²⁺], but also [Ca²⁺] itself by regulating the activity of Ca²⁺ pumps and channels, including the plasma membrane Ca²⁺-pumps, ryanodine receptors, IP3-receptors, and Ca²⁺-channels.

The NFAT family of transcription factors is composed of five proteins to date related to the Rel/NFκB family (NFATc1-c4 and NFAT5) (Hogan et al., (2003) Genes Dev 17:2205-2232). The characteristic feature of NFAT is its regulation by Ca²⁺ and the Ca²⁺/calmodulin-dependent serine-threonine phosphatase, calcineurin. In resting cells, NFAT proteins are phosphorylated and reside in the cytoplasm. Upon activation resulting from increases in intracellular Ca²⁺, the NFAT proteins are dephosphorylated by calcineurin, translocated to the nucleus, and become transcriptionally active, thus providing a direct link between intracellular Ca²⁺ signaling and gene expression (Hogan et al., (2003) Genes Dev 17:2205-2232).

Calcineurin is a heterodimer consisting of a catalytic subunit (CnA) and a regulatory subunit (CnB). The CnA subunit contains the phophatase domain, a CnB binding domain, a calmodulin binding domain, and an autoinhibitory loop. In resting cells, the autoinhibitory domain obscures the phophatase domain and is displaced upon binding of CnB and Ca2+/calmodulin to CnA resulting in the full activation of calcineurin. Calcineurin directly binds to and dephosphorylates NFATc1-c4 within the cytoplasm allowing them to translocated to the nucleus and participated in the regulation of gene expression (Bassel-Duby et al., (2003) Biochem Biophys Res Commun 311:1133-1141; Parsons et al., (2004) J Biol Chem 279:26192-26200; Crabtree & Olson, (2002) Cell 109 Suppl: S67-79). The Cn/NFAT signaling pathway is inhibited by CsA or FK506, potent immunosuppressive agents that are widely used to block the transcription of cytokine genes in activated T cells. These agents bind to cyclophilin and FK506 binding protein, respectively. The bound proteins then bind calcineurin, this inhibiting the desphophorylation and nuclear translocation of NFAT and T-cell activation.

Osteoblasts, during proliferation and differentiation, express several different genes that are regulated by various transcription factors that bind to specific response elements in the promoters of these genes. Due to the continuous identification of transcription and regulatory factors, the complexity of the molecular mechanisms that control gene expression and differentiation in osteoblasts are appreciated. It is shown herein that NFAT regulates osteoblast cellular differentiation

The oncoproteins, c-Fos and c-Jun, are the prototypical members of the AP-1 (activator protein 1) family of transcription factors, whose members also include FosB, Fra-1, Fra-2, c-Jun, JunB, and JunD. AP-1 is a dimeric transcription factor composed of either homodimers of the Jun family (c-Jun, JunB, and JunD) or heterodimers of the Jun and Fos family (c-Fos, Fra-1, Fra-2, and FosB) (Angel & Karin, (1991) Biochim Biophys Acta 1072:129-157) or the Jun and ATF family (ATF-2 ATF-3, and ATF-4) (Zayzafoon et al., (2002) J Biol Chem 277:37212-37218; Shaulian & Karin, (2001) Oncogene 20:2390-2400). Dimerization of AP-1 family members is a prerequisite for DNA binding to their 12-O-tetradecanoylphorbol 13-acetate (TPA) response elements (TRE) and CRE consensus elements (Shaulian & Karin, (2001) Oncogene 20:2390-2400; Ryseck & Bravo, (1991) Oncogene 6:533-542).

In osteoblasts, there is differential expression of AP-1 members during osteoblast growth and development (McCabe et al., (1996) Endocrinology 137:4398-4408). During osteoblast proliferation, all AP-1 members are expressed. The levels of these proteins decline as osteoblasts start to differentiate and enter the state of matrix maturation. During mineralization, Fra-2, and to a lesser extent, JunD are the major AP-1 members found in osteoblasts (McCabe et al., (1996) Endocrinology 137:4398-4408). Furthermore, antisense Fra-2 inhibits osteoblast differentiation and mineralization (McCabe et al., (1996) Endocrinology 137:4398-4408), and hormones such as PTH increase osteoblast differentiation by increasing Fra-2 expression (McCauley et al., (2001) Endocrinology 142:1975-1981). Analysis of the fra-2 promoter (Yoshida et al., (1993) Nucleic Acids Res 21:2715-2721) by a Transcription Element Search System program (Schug & Overton, (1997) Tech. Rep. CBIL-TR-1997-1001-v0.0) identified three potential NFAT consensus sequences. Two of these potential binding sites were located upstream of the transcriptional start site (−310 to −316 and −190 to −196). Interestingly, similarly to the CDK4 promoter, fra-2 has an NFAT binding site downstream of the transcriptional start site (+101 to +106). NFAT binding to this site could be responsible for the negative regulation of fra-2 expression.

Runx2 is an essential transcription factor for the differentiation of osteoblasts from mesenchymal precursors and the regulation of bone matrix deposition by differentiated osteoblasts. Homozygous Runx2-null mice show a complete lack of functional osteoblasts. Moreover, this factor is required for bone matrix synthesis by differentiated osteoblasts indicating that it regulates osteoblast gene expression at multiple levels. Furthermore, a dominant-negative Runx2 protein has been shown to prevent osteoblast differentiation in adult mice.

At the molecular level, Runx2 expression and activity are known to be modulated during the proliferation of osteoblast progenitors and the differentiation of osteoblasts by multiple transcriptional and post-translational mechanisms. Several osteoblast gene markers are regulated by Runx2, most importantly, osteocalcin. Osteocalcin expression is transcriptionally regulated by the binding of transcription factors to different regulatory elements identified in the proximal 800-bp region of its promoter. These regions include osteoblast specific elements 1 and 2 (OSE1, OSE2), AP-1/VDRE, and GRE. OSE1 and OSE2 were identified in the mouse osteocalcin promoter and are responsible for its specific activity in osteoblasts. Both of these cis-elements are occupied by osteoblast-specific transcription factors (Osf1) and Runx2, respectively. Interestingly, several transcription factors have been shown to interact physically with Runx2 leading to modulation of osteoblast differentiation and osteocalcin promoter activity. Furthermore, the immunoglobulin folds found in the DNA-binding domains of Runx2 are homologous with those found in NFAT. It appears that the Runx2 promoter has several NFAT binding sites, suggesting that NFAT binding may act as a repressor of Runx2 expression.

Secreted Wnt glycoproteins play a critical role in embryonic cell development, proliferation, and differentiation. Wnt proteins bind their receptors, frizzled and low-density lipoprotein receptor-related protein-5 or 6 (LRP-5/6), co-receptors and activate several signaling pathways, including the canonical β-catenin pathway. At a resting state, in the absence of Wnt/LRP activation, the phosphorylation of β-catenin by glycogen synthase kinase-3 (GSK-3) induces the degradation of β-catenin by the proteasome system. Upon activation by Wnt, GSK-3 is inhibited leading to the cytoplasmic accumulation of β-catenin which then translocates to the nucleus. There it associates with DNA binding proteins of the T-cell factor/lymphoid enhancer factor (TCF/LEF) family and activates gene transcription. Osteoblast differentiation is orchestrated by a finite number of transcription factors and regulators, one of which is β-catenin. Human and mouse genetic studies demonstrate that the β-catenin signaling pathway is critical for bone formation.

Osteoblasts originate from mesenchymal progenitors or osteoprogenitor cells that, with the appropriate stimulation, undergo proliferation and differentiate into preosteoblasts and then into mature, functional osteoblasts. In culture, as in vivo, osteoblasts form bone-like mineralized nodules by undergoing three stages of development; proliferation, extracellular matrix maturation, and mineralization. During each stage of development, specific subsets of genes are sequentially expressed or repressed. For example, collagen I is known to be a marker for proliferation, alkaline phosphatase for extracellular matrix maturation, and osteocalcin for mineralization. The regulation of gene expression in osteoblasts during development and differentiation occurs predominantly at the transcriptional level. Several transcription factors and signaling pathways, such as AP-1, Runx2, and β-catenin have been shown to play a major role in the regulation of osteoblast gene expression, phenotype, and ultimately bone formation.

As used herein, the term “osteoblast cell” refers to a terminally or non-terminally differentiated cell derived from a bone precursor cell, wherein the osteoblast cell is at least more differentiated towards an osteoblast phenotype than the cell from which it is derived. As used herein, “osteoblast cells” are characterized by the expression of one or more specific marker transcripts, such as, but not limited to, AP-1 family members, Runx2, Fra-2, alkaline phosphatase, osteocalcin, β-catenin, CCAAT/enhancer binding protein (C/EBP), and ATF4, and may also show matrix deposition, matrix mineralization, and/or cuboidal morphology of the cells. Furthermore, as used herein, the term “terminally differentiated osteoblast” refers to an osteoblast cell that is actively producing and mineralizing bone material.

Also, as used herein, producing an osteoblast cell encompasses the production of a cell culture that is enriched for osteoblast cells. In certain embodiments of the present invention, the term “enriched” refers to a cell culture that contains more than approximately 50%, 55%, 60%, 65%, 70%, 75%, 80%, 85%, 90%, or 95% of the desired cell lineage.

As used herein, the term “bone precursor cell” refers to a cell that differentiates towards the osteoblast lineage upon treatment with known osteoblast-promoting agents, such as, but not limited to type I collagen, fibrinogen, fibrin, fibrinogen, osteocalcin, osteonectin, TGF-β, 1,25-OH Vitamin D3, basic fibroblast growth factor, or bone morphogenic protein 2. It is preferred that the bone precursor cell express one or more of osteocalcin, osteonectin or alkaline phosphatase. In a preferred embodiment, bone precursor cells include osteoprogenitor cells or preosteoblasts.

As used herein, the term “differentiate” refers to the production of a cell type that is more differentiated than the cell type from which it is derived. The term therefore encompasses cell types that are partially and terminally differentiated.

As used herein, the terms “biologically active compound” or “bioactive component” and “bioactive factor” refer to any compound or molecule that induces a progenitor cell to partially or terminally differentiate, wherein said differentiation is due at least in part to modulation of signaling through the calcineurin and/or NFAT signaling pathway. While the bioactive compound may be as described below, the term is not limited thereto. The term “bioactive component” as used herein includes within its scope a natural or synthetic molecule or molecules which exhibit(s) similar biological activity.

As used herein, the term “member of the NFAT superfamily” refers to transcription factors that are generally characterized by one of skill in the art as belonging to the NFAT superfamily, either due to homology with known members of the NFAT superfamily, or due to similarity in function with known members of the NFAT superfamily. In certain embodiments, the member of the NFAT superfamily is selected from the group consisting of NFATc1, NFATc2, NFATc3, NFATc4, or NFAT5. In one embodiment, the member of the NFAT superfamily is selected from NFATc1 and NFATc3. In a further embodiment, the member of the NFAT superfamily is NFATc1.

In a further embodiment, the activity of the NFAT superfamily member is inhibited by administration of an inactivator of the calcineurin signaling pathway. As used herein, the term “inactivator of the calcineurin signaling pathway” refers to any molecule or compound that decreases the activity of calcineurin or modulates the activity of at least one molecule downstream of calcineurin in a cell contacted with the inactivator. It is understood that combinations of inactivators may be used to elicit the desired effect. Calcineurin dephosphorylates other molecules, among which include the NFAT superfamily. An inactivator of calcineurin signaling can decrease the dephosphorylation and nuclear translocation of one or more members of the NFAT superfamily. Preferably the members of the NFAT superfamily are transcription factors. Therefore, inactivating calcineurin signaling may result in the increase or decrease of downstream signaling molecules. It is contemplated that the inactivator of calcineurin signaling may act directly on calcineurin or may act on a molecule upstream or downstream of calcineurin to thereby inactivate calcineurin signaling. In certain embodiments, the inactivator of the calcineurin signaling pathway is selected from the group consisting of CsA and FK506.

In one embodiment, the target cells are contacted with an effective amount of an inactivator of the calcineurin signaling pathway. As used herein, the term “effective amount” of an inactivator of the calcineurin signaling pathway refers to that concentration of the compound that is sufficient to effect differentiation of a target cell towards a desired cell lineage, preferably, towards an osteoblast lineage. In one embodiment, the effective amount of an inactivator of the calcineurin signaling pathway ranges from approximately 1 nM to approximately 50 μM. If the inactivator of the calcineurin signaling pathway is CsA, the effective amount is from approximately 1 nM to approximately 10 μM, or more preferably, from between approximately 1 nM and approximately 1 μM. If the inactivator of the calcineurin signaling pathway is FK506, the effective amount is from approximately 25 nM to approximately 50 μM, more preferably, from between approximately 500 nM and approximately 25 μM, or more preferably, from between approximately 500 nM and approximately 10 μM. In certain embodiments, using amounts higher than the effective amounts of the inactivator of the calcineurin signaling pathway can inhibit osteoblast differentiation. These concentrations are readily determined by one of ordinary skill in the art.

As used herein when referring to a cell, cell line, cell culture or population of cells, the term “isolated” refers to being substantially separated from the natural source of the cells such that the cell, cell line, cell culture, or population of cells are capable of being cultured in vitro. In addition, the term “isolating” is used to refer to the physical selection of one or more cells out of a group of two or more cells, wherein the cells are selected based on cell morphology and/or the expression of various markers.

As used herein, the term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, RT-PCT, in situ hybridization, Western blotting, and immunostaining.

As used herein, the term “contacting” (i.e., contacting a cell e.g. a target cell, with a compound) is intended to include incubating the compound and the cell together in vitro (e.g., adding the compound to cells in culture). The term “contacting” is not intended to include the in vivo exposure of cells to an inactivator of the calcineurin signaling pathway that may occur naturally in a subject (i.e., exposure that may occur as a result of a natural physiological process). The step of contacting the cell with a test compound can be conducted in any suitable manner. For example, the cells may be treated in adherent culture or in suspension culture. It is understood that the cells contacted with an inactivator of the calcineurin signaling pathway may be further treated with other cell differentiation environments to stabilize the cells, or to differentiate the cells further.

A cell differentiating medium or environment may be utilized to partially, terminally, or reversibly differentiate the bone progenitor cells of the present invention, either prior to, during, or after contacting the bone progenitor cells with an inactivator of the calcineurin signaling pathway. In accordance with the invention the medium of the cell differentiation environment may contain a variety of components including, for example, DMEM, Ham's F12 medium, FBS (fetal bovine serum), FGF2 (fibroblast growth factor), α-MEM, vitamin C, beta glycerophosphate. The cell differentiation environment can also contain supplements such as L-Glutamine, NEAA (non-essential amino acids), and P/S (penicillin/streptomycin). It is contemplated that additional factors may be added to the cell differentiation environment, including, but not limited to, fibronectin, laminin, heparin, heparin sulfate, retinoic acid, members of the epidermal growth factor family (EGFs), members of the fibroblast growth factor family (FGFs) including FGF2 and/or FGF8, members of the platelet derived growth factor family (PDGFs), transforming growth factor (TGF)/bone morphogenetic protein (BMP)/growth and differentiation factor (GDF) factor family antagonists including but not limited to noggin, follistatin, chordin, gremlin, cerberus/DAN family proteins, ventropin, high dose activin, and amnionless. TGF/BMP/GDF antagonists could also be added in the form of TGF/BMP/GDF receptor-Fc chimeras. Other factors that may be added include molecules that can activate or inactivate signaling through Notch receptor family, including but not limited to proteins of the Delta-like and Jagged families as well as inhibitors of Notch processing or cleavage. Other growth factors may include members of the insulin like growth factor family (IGF), insulin, the wingless related (WNT) factor family, and the hedgehog factor family.

In other embodiments, the cell differentiation environment comprises plating the cells in an adherent culture. As used herein, the terms “plated” and “plating” refer to any process that allows a cell to be grown in adherent culture. As used herein, the term “adherent culture” refers to a cell culture system whereby cells are cultured on a solid surface, which may in turn be coated with a solid substrate that may in turn be coated with another surface coat of a substrate, such as those listed below, or any other chemical or biological material that allows the cells to proliferate or be stabilized in culture. The cells may or may not tightly adhere to the solid surface or to the substrate. In one embodiment, the cells are plated on matrigel coated plates. The substrate for the adherent culture may comprise any one or combination of polyornithine, laminin, poly-lysine, purified collagen, gelatin, extracellular matrix, fibronectin, tenascin, vitronectin, entactin, heparin sulfate proteoglycans, poly glycolytic acid (PGA), poly lactic acid (PLA), poly lactic-glycolic acid (PLGA) and feeder layers such as, but not limited to, primary fibroblasts or fibroblast cells lines. Furthermore, the substrate for the adherent culture may comprise the extracellular matrix laid down by a feeder layer, or laid down by the target cell or cell culture.

The methods of the present invention contemplate that target cells may be cultured with a feeder cell or feeder layer. As used herein, a “feeder cell” is a cell that is co-cultured with a target cell and stabilizes the target cell in its current state of differentiation. A feeder layer comprises more than one feeder cell in culture. In one embodiment of the above method, conditioned medium is obtained from a feeder cell that stabilizes the target cell in its current state of differentiation. Any and all factors produced by a feeder cell that allow a target cell to be stabilized in its current state of differentiation can be isolated and characterized using methods routine to those of skill in the art. These factors may be used in lieu of a feeder layer, or may be used to supplement a feeder layer.

As used herein, the term “stabilize” refers to the differentiation state of a cell. When a cell or cell population is stabilized, it will continue to proliferate over multiple passages in culture, and preferably indefinitely in culture; additionally, each cell in the culture is preferably of the same differentiation state, and when the cells divide, typically yield cells of the same cell type or yield cells of the same differentiation state. Preferably, a stabilized cell or cell population does not further differentiate or de-differentiate if the cell culture conditions are not altered, and the cells continue to be passaged and are not overgrown. Preferably the cell that is stabilized is capable of proliferation in the stable state indefinitely, or for at least more than 2 passages. Preferably, it is stable for more than 5 passages, more than 10 passages, more than 15 passages, more than 20 passages, more than 25 passages, or most preferably, it is stable for more than 30 passages. In one embodiment, the cell is stable for greater than 1 year of continuous passaging.

With respect to some of the embodiments of differentiation methods described herein, the above-mentioned growth factors are provided to the cells so that the growth factors are present in the cultures at concentrations sufficient to promote differentiation of at least a portion of the target cells to the desired cell lineage. In some embodiments of the present invention, the above-mentioned growth factors are present in the cell culture at a concentration of at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml.

In certain embodiments of the present invention, the above-mentioned growth factors are removed from the cell culture subsequent to their addition. For example, the growth factors can be removed within about one day, about two days, about three days, about four days, about five days, about six days, about seven days, about eight days, about nine days or about ten days after their addition.

The compositions and methods described herein have several useful features. For example, the compositions and methods described herein are useful for modeling the stages of bone development. Furthermore, the compositions and methods described herein can also serve for therapeutic intervention in disease states, such as osteoporosis, osteopenia, or other bone-loss or bone density decreasing disorders. For example, since osteoblast-progenitor cells serve as the source for only a limited number tissues, it can be used in the development of pure tissue or cell types. In addition, compounds that inhibit the activity of calcineurin and/or inhibit the dephosphorylation and/or nuclear translocation of a member of the NFAT superfamily can be formulated into a pharmaceutical formulation for the treatment of a disease state, such as, but not limited to osteoporosis, osteopenia, or other bone-loss or bone density decreasing disorders.

The cell types that differentiate from bone precursor cells after contact with an inactivator of the calcineurin and or NFAT signaling pathway have several uses in various fields of research and development including but not limited to drug discovery, drug development and testing, toxicology, production of cells for therapeutic purposes as well as basic science research. These cell types express molecules that are of interest in a wide range of research fields. These include the molecules known to be required for the functioning of the various cell types as described in standard reference texts. These molecules include, but are not limited to, cytokines, growth factors, cytokine receptors, extracellular matrix, transcription factors, secreted polypeptides and other molecules, and growth factor receptors. In addition, the cells can be used as a source of nuclear material for nuclear transfer techniques and used to produce cells, tissues or components of organs for transplant. The test compounds that increase differentiation of osteoblasts also have a number of functions, including but not limited to the treatment of various bone disorders, usefulness in determining the molecular signaling pathways involved in bone development, demineralization and bone regrowth.

The progression of the target cell culture to the desired cell lineage can be monitored by quantitating expression of marker genes characteristic of the desired cell lineage as well as the lack of expression of marker genes characteristic of bone progenitor cells and other cell types. One method of quantitating gene expression of such marker genes is through the use of quantitative PCR (Q-PCR). Methods of performing Q-PCR are well known in the art. Other methods that are known in the art can also be used to quantitate marker gene expression. Marker gene expression can be detected by using antibodies specific for the marker gene of interest.

In some embodiments of the present invention, cells of the desired cell lineage can be isolated by using an affinity tag that is specific for such cells. One example of an affinity tag specific for a target cell is an antibody that is specific to a marker polypeptide that is present on the cell surface of the target cell but which is not substantially present on other cell types that would be found in a cell culture produced by the methods described herein.

As described above, the invention encompasses a method of improving bone mass in an individual having a bone-related disorder, by administering to the individual a therapeutically effective amount of a compound. As used herein, the phrase “bone-related disorder” refers to a disorder wherein bone formation, deposition, or resorption is abnormal. Bone-related disorders include, but are not limited to, osteoporosis, bone fractures, hypercalcemia of malignancy, osteopenia or osteolytic lesions due to bone metastases, periprosthetic osteolysis, periodontal disease, tooth loss, rheumatoid arthritis, osteoarthritis, hyperparathyroidism, Paget's disease, osteodystrophy, myositis ossificans, Bechterew's disease, malignant hypercalcernia, bone loss, bone abnormalities due to steroid hormone treatment, bone abnormalities caused by cancer therapeutics, abnormally increased bone turnover, osteomalacia, Bechet's disease, hyperostosis, osteopetrosis, osteogenesis imperfecta, rachitis, immobilization-induced osteopenia, and glucocorticoid-induced osteoporosis.

Another aspect of this invention is directed to methods for strengthening a bone graft, inducing vertebral synostosis, enhancing long bone extension, the treatment and promotion of healing of bone fractures and osteotomies, enhancing bone healing following facial reconstruction, maxillary reconstruction and/or mandibular reconstruction in a vertebrate, e.g., a mammal (including a human being), comprising administering to said vertebrate a therapeutically effective amount of a compound of the current invention, a prodrug or a pharmaceutically acceptable salt thereof, or a stereoisomer or diastereomeric mixture of said compound, prodrug or salt. The composition may be applied locally to the site of bone reconstruction or may be administered systemically.

Administration of the compounds of this invention can be via any mode that delivers the compound systemically and/or locally (e.g., at the site of the bone fracture, osteotomy, or orthopedic surgery).

In the methods of the present invention, the compounds described herein and determined using the screening methods described herein, can form the active ingredient, and are typically administered in admixture with suitable pharmaceutically acceptable diluents, excipients, adjuvants or carriers (collectively referred to herein as “carrier” materials) suitably selected with respect to the intended form of administration, that is, oral tablets, capsules, pills, powders, granules, elixirs, tinctures, suspensions, syrups and the like, and consistent with conventional pharmaceutical practices. Likewise, they may also be administered in intravenous (bolus or infusion), intraperitoneal, intranasal, rectal, topical, subcutaneous, intramuscular or transdermal form, all using forms well known to those of ordinary skill in the pharmaceutical arts.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

The compounds can be applied to the sites of bone fractures or osteotomies, for example, either by injection of the compound in a suitable solvent (e.g., an oily solvent such as arachis oil) to the cartilage growth plate or, in cases of open surgery, by local application thereto of the compound in a suitable vehicle, carrier or diluent such as bone-wax, demineralized bone powder, polymeric bone cements, bone sealants, etc. Alternatively, local application can be achieved by applying a solution or dispersion of the compound in a suitable carrier or diluent onto the surface of, or incorporating it into solid or semi-solid implants conventionally used in orthopedic surgery, such as dacron-mesh, gel-foam and kiel bone, or prostheses.

As used herein, the phrase “pharmaceutically acceptable” refers to an agent that does not interfere with the effectiveness of the biological activity of an active ingredient, and which may be approved by a regulatory agency of the Federal government or a state government, or is listed in the U.S. Pharmacopeia or other generally recognized pharmacopeia for use in animals, and more particularly for use in humans. Accordingly, suitable pharmaceutically acceptable carriers include agents that do not interfere with the effectiveness of a pharmaceutical composition.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

Methods of preparing various pharmaceutical compositions with a certain amount of active ingredient are known to those skilled in the art. For examples of methods of preparing pharmaceutical compositions, see Remington: The Science and Practice of Pharmacy, Mack Publishing Company, Easton, Pa., 19th Edition (1995).

The instant compounds are also useful in combination with known agents useful for treating bone-related disorders. Combinations of the presently disclosed compounds with other agents useful in treating osteoporosis or other bone disorders are within the scope of the invention. A person of ordinary skill in the art would be able to discern which combinations of agents would be useful based on the particular characteristics of the drugs and the disease involved. Such agents include but are not limited to the following: an organic bisphosphonate; a cathepsin K inhibitor; an estrogen or an estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; calcitonin; Vitamin D or a synthetic Vitamin D analogue; selective serotonin reuptake inhibitors (SSRIs); and the pharmaceutically acceptable salts and mixtures thereof.

The term “administration” and variants thereof (e.g., “administering” a compound) in reference to a compound of the invention means introducing the compound or a prodrug of the compound into the system of the individual in need of treatment. When a compound of the invention or prodrug thereof is provided in combination with one or more other active agents (e.g., a bisphosphonate, etc.), “administration” and its variants are each understood to include concurrent and sequential introduction of the compound or prodrug thereof and other agents.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

When a compound according to this invention is administered into a human subject, the daily dosage will normally be determined by the prescribing physician with the dosage generally varying according to the age, sex, weight, and response of the individual patient, as well as the severity of the patient's symptoms, the route of administration; and the particular compound or salt thereof employed. An ordinarily skilled physician, veterinarian or clinician can readily determine and prescribe the effective amount of the drug required to prevent, counter or arrest the progress of the condition.

In one exemplary application, a suitable amount of compound is administered to a mammal undergoing treatment. Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The compositions and methods of the present invention are administered and carried out until the desired therapeutic effect is achieved. The term “until the desired therapeutic effect is achieved”, as used herein, means that the therapeutic agent or agents are continuously administered, according to the dosing schedule chosen, up to the time that the clinical or medical effect sought for the disease or condition being treated is observed by the clinician or researcher. For methods of treatment of the present invention, the pharmaceutical composition is continuously administered until the desired improvement in bone mass or structure is observed. In such instances, achieving an improvement in bone mass or a replacement of abnormal bone structure with normal bone structure are the desired objectives. For methods of prevention of the present invention, the pharmaceutical composition is continuously administered for as long as necessary to prevent the undesired condition. In such instances, maintenance of bone mass density is often the objective. Non-limiting examples of administration periods can range from about 2 weeks to the remaining lifespan of the mammal. For humans, administration periods can range from about 2 weeks to the remaining lifespan of the human, preferably from about 2 weeks to about 20 years, more preferably from about 1 month to about 20 years, more preferably from about 6 months to about 10 years, and most preferably from about 1 year to about 10 years.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

As used herein, the term “improving” with respect to bone mass includes increasing or maintaining the current bone mass of an individual, and includes slowing the rate of bone loss. As such, the term reducing or inhibiting the resorption of bone in bone-related disorders. As described herein, determining the nuclear translocation of a member of the NFAT superfamily, the dephosphorylation of a calcineurin substrate, or expression of an osteoblast specific marker transcript in a bone precursor cell upon in vitro contact with a compound is predictive that the compound is useful for treating a bone-related disorder, or improving bone mass.

The term “bone resorption,” as used herein, refers to the process by which osteoclasts degrade bone.

As used herein, the term “bone mass” refers to bone mass per unit area, which is sometimes referred to as bone mineral density.

In the present invention the compounds can be used to inhibit bone resorption, or more specifically to inhibit undesired or abnormal bone resorption. The term “abnormal bone resorption”, as used herein means a degree of bone resorption that exceeds the degree of bone formation, either locally, or in the skeleton as a whole. Alternatively, “abnormal bone resorption” can be associated with the formation of bone having an abnormal structure, as in Paget's disease.

The term “bone resorption inhibiting”, as used herein, means preventing bone resorption by the direct or indirect alteration of osteoclast formation or activity. Inhibition of bone resorption refers to prevention of bone loss, especially the inhibition of removal of existing bone either from the mineral phase and/or the organic matrix phase, through direct or indirect alteration of osteoclast formation or activity.

As used herein, the term “nucleic acid” and “polynucleotide” refer to RNA or DNA that is linear or branched, single or double stranded, or a hybrid thereof. The term also encompasses RNA/DNA hybrids. These terms also encompass untranslated sequence located at both the 3′ and 5′ ends of the coding region of the gene: at least about 1000 nucleotides of sequence upstream from the 5′ end of the coding region and at least about 200 nucleotides of sequence downstream from the 3′ end of the coding region of the gene. Less common bases, such as inosine, 5-methylcytosine, 6-methyladenine, hypoxanthine, and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The antisense polynucleotides and ribozymes can consist entirely of ribonucleotides, or can contain mixed ribonucleotides and deoxyribonucleotides. The polynucleotides of the invention may be produced by any means, including genomic preparations, cDNA preparations, in vitro synthesis, RT-PCR, and in vitro or in vivo transcription.

An “isolated” nucleic acid molecule is one that is substantially separated from other nucleic acid molecules, which are present in the natural source of the nucleic acid (i.e., sequences encoding other polypeptides). Preferably, an “isolated” nucleic acid is free of some of the sequences, which naturally flank the nucleic acid (i.e. sequences located at the 5′ and 3′ ends of the nucleic acid) in its naturally occurring replicon. For example, a cloned nucleic acid is considered isolated. In various embodiments, the isolated nucleic acid molecule can contain less than about 5 kb, 4 kb, 3 kb, 2 kb, 1 kb, 0.5 kb, or 0.1 kb of nucleotide sequences which naturally flank the nucleic acid molecule in genomic DNA of the cell from which the nucleic acid is derived. A nucleic acid is also considered isolated if it has been altered by human intervention, or placed in a locus or location that is not its natural site, or if it is introduced into a cell by transfection. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

Specifically excluded from the definition of “isolated nucleic acids” are: naturally-occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein a specified nucleic acid makes up less than 5% of the number of nucleic acid inserts in the vector molecules. Further specifically excluded are whole cell genomic DNA or whole cell RNA preparations (including whole cell preparations that are mechanically sheared or enzymatically digested). Even further specifically excluded are the whole cell preparations found as either an in vitro preparation or as a heterogeneous mixture separated by electrophoresis wherein the nucleic acid of the invention has not further been separated from the heterologous nucleic acids in the electrophoresis medium (e.g., further separating by excising a single band from a heterogeneous band population in an agarose gel or nylon blot).

Nucleic acid molecules can be isolated using standard molecular biology techniques and the sequence information provided herein. For example, mRNA can be isolated from a cell, and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed. A nucleic acid molecule can be amplified using cDNA or, alternatively, genomic DNA, as a template and appropriate oligonucleotide primers according to standard PCR amplification techniques. The nucleic acid molecule so amplified can be cloned into an appropriate vector and characterized by DNA sequence analysis. Furthermore, oligonucleotides corresponding to a known nucleotide sequence can be prepared by standard synthetic techniques, e.g., using an automated DNA synthesizer.

In addition to fragments and fusion polypeptides of the nucleic acid molecules, the present invention includes homologs and analogs of naturally occurring polypeptides. “Homologs” are defined herein as two nucleic acids or polypeptides that have similar, or “identical,” nucleotide or amino acid sequences, respectively. Homologs include allelic variants, orthologs, paralogs, agonists, and antagonists of naturally occurring nucleic acids as defined hereafter. The term “homolog” further encompasses nucleic acid molecules that differ from the determined nucleotide sequence due to degeneracy of the genetic code and thus encode the same polypeptide. As used herein, a “naturally occurring” polypeptide refers to an amino acid sequence that occurs in nature.

An agonist of a polypeptide can retain substantially the same, or a subset, of the biological activities of the polypeptide. An antagonist of a polypeptide can inhibit one or more of the activities of the naturally occurring form of the polypeptide.

Nucleic acid molecules corresponding to natural allelic variants and analogs, orthologs, and paralogs of a nucleic acid sequence can be isolated based on their identity to the known nucleic acids, or a portion thereof, as a hybridization probe according to standard hybridization techniques under stringent hybridization conditions. In an alternative embodiment, homologs of the nucleic acid sequence can be identified by screening combinatorial libraries of mutants, e.g., truncation mutants, for agonist or antagonist activity.

To determine the percent sequence identity of two amino acid sequences, the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence is occupied by the same amino acid residue as the corresponding position in the other sequence, then the molecules are identical at that position. The same type of comparison can be made between two nucleic acid sequences.

The percent sequence identity between the two sequences is a function of the number of identical positions shared by the sequences (i.e., percent sequence identity=numbers of identical positions/total numbers of positions×100). Preferably, the isolated amino acid homologs are at least about 50-60%, preferably at least about 60-70%, and more preferably at least about 70-75%, 75-80%, 80-85%, 85-90%, or 90-95%, and most preferably at least about 96%, 97%, 98%, 99%, or more identical.

For the purposes of the invention, the percent sequence identity between two nucleic acid or polypeptide sequences is determined using the Vector NTI 6.0 (PC) software package (InforMax, 7600 Wisconsin Ave., Bethesda, Md. 20814). A gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

As used herein with regard to hybridization for DNA to a DNA blot, the term “stringent conditions” may refer to hybridization overnight at 60 C in 10× Denhardt's solution, 6×SSC, 0.5% SDS, and 100 μg/ml denatured salmon sperm DNA. Blots are washed sequentially at 62 C for 30 minutes each time in 3×SSC/0.1% SDS, followed by 1×SSC/0.1% SDS, and finally 0.1×SSC/0.1% SDS. In a preferred embodiment, the phrase “stringent conditions” refers to hybridization in a 6×SSC solution at 65 C. As also used herein, “highly stringent conditions” refers to hybridization overnight at 65 C in 10× Denhardt's solution, 6×SSC, 0.5% SDS, and 100 μg/ml denatured salmon sperm DNA. Blots are washed sequentially at 65 C for 30 minutes each time in 3×SSC/0.1% SDS, followed by 1×SSC/0.1% SDS, and finally 0.1×SSC/0.1% SDS. Methods for nucleic acid hybridizations are described in Meinkoth and Wahl, 1984, Anal. Biochem. 138:267-284; Current Protocols in Molecular Biology, Chapter 2, Ausubel et al. Eds., Greene Publishing and Wiley-Interscience, New York, 1995; and Tijssen, 1993, Laboratory Techniques in Biochemistry and Molecular Biology: Hybridization with Nucleic Acid Probes, Part I, Chapter 2, Elsevier, New York, 1993.

Using the above-described methods, and others known to those of skill in the art, one of ordinary skill in the art can isolate homologs of known nucleic acid sequences. One subset of these homologs is allelic variants. As used herein, the term “allelic variant” refers to a nucleotide sequence containing polymorphisms that lead to changes in the amino acid sequences and that exist within a natural population. Such natural allelic variations can typically result in 1-5% variance in a nucleic acid.

Moreover, nucleic acid molecules encoding a polypeptide from the same or other species such as analogs, orthologs, and paralogs, are intended to be within the scope of the present invention. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. Normally, orthologs encode polypeptides having the same or similar functions. As also used herein, the term “paralogs” refers to two nucleic acids that are related by duplication within a genome. Paralogs usually have different functions, but these functions may be related (Tatusov, R. L. et al., 1997, Science 278 (5338):631-637).

In addition to naturally-occurring variants of a sequence that may exist in the population, the skilled artisan will further appreciate that changes can be introduced by mutation into a nucleotide sequence, thereby leading to changes in the amino acid sequence of the encoded protein, without altering the functional activity of the molecule. For example, nucleotide substitutions leading to amino acid substitutions at “non-essential” amino acid residues can be made in a sequence. A “non-essential” amino acid residue is a residue that can be altered from the wild-type sequence without altering the activity of said protein, whereas an “essential” amino acid residue is required for the activity. Other amino acid residues, however, (e.g., those that are not conserved or only semi-conserved in a domain having biological activity) may not be essential for activity and thus are likely to be amenable to alteration without altering activity. One or more amino acid substitutions, additions, or deletions can be introduced into the encoded polypeptide by mutating the nucleic acid using standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain.

Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Thus, a predicted nonessential amino acid residue is preferably replaced with another amino acid residue from the same side chain family. Alternatively, in another embodiment, mutations can be introduced randomly along all or part of a coding sequence, such as by saturation mutagenesis, and the resultant mutants can be screened for biological activity described herein to identify mutants that retain or do not retain specific biological activity

Antisense polynucleotides are thought to inhibit gene expression of a target polynucleotide by specifically binding the target polynucleotide and interfering with transcription, splicing, transport, translation, and/or stability of the target polynucleotide. Methods are described in the prior art for targeting the antisense polynucleotide to the chromosomal DNA, to a primary RNA transcript, or to a processed mRNA. Preferably, the target regions include splice sites, translation initiation codons, translation termination codons, and other sequences within the open reading frame.

The term “antisense,” for the purposes of the invention, refers to a nucleic acid comprising a polynucleotide that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. The term “antisense nucleic acid” includes single stranded RNA as well as double-stranded DNA expression cassettes that can be transcribed to produce an antisense RNA. “Active” antisense nucleic acids are antisense RNA molecules that are capable of selectively hybridizing with a primary transcript or mRNA encoding a polypeptide having at least 80% sequence identity with the targeted polypeptide sequence.

The antisense nucleic acid can be complementary to an entire coding strand, or to only a portion thereof. In one embodiment, an antisense nucleic acid molecule is antisense to a “coding region” of the coding strand of a nucleotide sequence. The term “coding region” refers to the region of the nucleotide sequence comprising codons that are translated into amino acid residues. In another embodiment, the antisense nucleic acid molecule is antisense to a “noncoding region” of the coding strand of a nucleotide sequence. The term “noncoding region” refers to 5′ and 3′ sequences that flank the coding region that are not translated into amino acids (i.e., also referred to as 5′ and 3′ untranslated regions). The antisense nucleic acid molecule can be complementary to the entire coding region of mRNA, but more preferably is an oligonucleotide that is antisense to only a portion of the coding or noncoding region of an mRNA. For example, the antisense oligonucleotide can be complementary to the region surrounding the translation start site. An antisense oligonucleotide can be, for example, about 5, 10, 15, 20, 25, 30, 35, 40, 45, or 50 nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids. Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The antisense nucleic acid molecules of the invention are typically administered to a cell or generated in situ such that they hybridize with or bind to cellular mRNA and/or genomic DNA to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic promoter are preferred.

The present invention further provides compositions for RNA interference. In this technique, double-stranded RNA or dsRNA derived from the gene to be analyzed is introduced into the target cell. As used herein, “dsRNA” refers to RNA that is partially or completely double stranded. The dsRNA may have a single stranded overhang at either or both ends of the molecule. This dsRNA is processed into relatively small fragments and can subsequently become distributed throughout the nematode. The dsRNA fragments interact, in a cell, with the corresponding endogenously produced messenger RNA, resulting in the endogenous transcript being specifically broken down (Zamore et al., 2000 Cell 101:25-33). This process leads to a loss-of-function mutation having a phenotype that, over the period of a generation, may come to closely resemble the phenotype arising from a complete or partial deletion of the target gene.

The invention provides for a composition comprising a dsRNA that is substantially identical to a portion of a target gene of the target cell genome. In certain embodiments of the foregoing, the target gene is selected from the group consisting of (a) the polynucleotide sequence encoding NFATc1, and the polynucleotide sequence encoding NFATc3, and (b) a polynucleotide that hybridizes under stringent conditions to a polynucleotide as defined in (a). The polynucleotide and polypeptide sequences encoding human NFATc1 are available at GeneID number 4772. The polynucleotide and polypeptide sequences encoding mouse NFATc1 are available at GeneID number 18018. The polynucleotide and polypeptide sequences encoding human NFATc3 are available at GeneID number 4775. The polynucleotide and polypeptide sequences encoding mouse NFATc3 are available at GeneID number 18021.

The invention further provides for a composition comprising a dsRNA consisting of (a) a first stand comprising a sequence substantially identical to 19-49 consecutive nucleotides of the polynucleotide sequence encoding NFATc1, or the polynucleotide sequence encoding NFATc3; and (b) a second strand comprising a sequence substantially complementary to the first strand. Preferably, the dsRNA inhibits expression of a protein encoded by a polynucleotide hybridizing under stringent conditions to the polynucleotide sequence encoding NFATc1, or the polynucleotide sequence encoding NFATc3. In further embodiments, the dsRNA has a single stranded overhang at either or both ends. The invention provides for a nucleic acid molecule comprising a regulatory sequence operatively linked to a nucleotide sequence that is a template for one or both strands of the claimed dsRNA. In one embodiment, the nucleic acid molecule further comprises a promoter flanking either end of the nucleic acid molecule, wherein the promoters drive expression of each individual DNA strand, thereby generating two complementary RNAs that hybridize and form the dsRNA. In another embodiment, the nucleic acid molecule comprises a nucleotide sequence that is transcribed into both strands of the dsRNA on one transcription unit, wherein the sense strand is transcribed from the 5′ end of the transcription unit and the antisense strand is transcribed from the 3′ end, wherein the two strands are separated by 3 to 500 base pairs, and wherein after transcription, the RNA transcript folds on itself to form a hairpin.

As an alternative to antisense polynucleotides, ribozymes, sense polynucleotides, or double stranded RNA (dsRNA) can be used to reduce expression of a polypeptide. As used herein, the term “ribozyme” refers to a catalytic RNA-based enzyme with ribonuclease activity that is capable of cleaving a single-stranded nucleic acid, such as an mRNA, to which it has a complementary region. Ribozymes (e.g., hammerhead ribozymes described in Haselhoff and Gerlach, 1988, Nature 334:585-591) can be used to catalytically cleave mRNA transcripts to thereby inhibit translation. A ribozyme having specificity for a nucleic acid can be designed based upon the nucleotide sequence of the cDNA or on the basis of a heterologous sequence to be isolated according to methods taught in this invention. In preferred embodiments, the ribozyme will contain a portion having at least 7, 8, 9, 10, 12, 14, 16, 18, or 20 nucleotides, and more preferably 7 or 8 nucleotides, that have 100% complementarity to a portion of the target RNA. Methods for making ribozymes are known to those skilled in the art. See, e.g., U.S. Pat. Nos. 6,025,167; 5,773,260; and 5,496,698.

The term “dsRNA,” as used herein, refers to RNA hybrids comprising two strands of RNA. The dsRNAs can be linear or circular in structure. The hybridizing RNAs may be substantially or completely complementary. By “substantially complementary,” is meant that when the two hybridizing RNAs are optimally aligned using the BLAST program as described above, the hybridizing portions are at least 95% complementary. Preferably, the dsRNA will be at least 100 base pairs in length. Typically, the hybridizing RNAs will be of identical length with no over hanging 5′ or 3′ ends and no gaps. However, dsRNAs having 5′ or 3′ overhangs of up to 100 nucleotides may be used in the methods of the invention.

The dsRNA may comprise ribonucleotides, ribonucleotide analogs such as 2′-O-methyl ribosyl residues, or combinations thereof. See, e.g., U.S. Pat. Nos. 4,130,641 and 4,024,222. A dsRNA polyriboinosinic acid:polyribocytidylic acid is described in U.S. Pat. No. 4,283,393. Methods for making and using dsRNA are known in the art.

A useful method to ascertain the level of transcription of the gene (an indicator of the amount of mRNA available for translation to the gene product) is to perform a Northern blot (For reference, see, for example, Ausubel et al., 1988, Current Protocols in Molecular Biology, Wiley: New York). The information from a Northern blot at least partially demonstrates the degree of transcription of the transformed gene. Total cellular RNA can be prepared from cells, tissues, or organs by several methods, all well-known in the art, such as that described in Bormann, E. R. et al., 1992, Mol. Microbiol. 6:317-326. To assess the presence or relative quantity of polypeptide translated from this mRNA, standard techniques, such as a Western blot, may be employed. These techniques are well known to one of ordinary skill in the art. (See, for example, Ausubel et al., 1988, Current Protocols in Molecular Biology, Wiley: New York).

As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses, and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. As used herein with respect to a recombinant expression vector, “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990), including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides.

Another aspect of the invention pertains to isolated polypeptides, and biologically active portions thereof. An “isolated” or “purified” polypeptide or biologically active portion thereof is free of some of the cellular material when produced by recombinant DNA techniques, or chemical precursors or other chemicals when chemically synthesized. The language “substantially free of cellular material” includes preparations in which the polypeptide is separated from some of the cellular components of the cells in which it is naturally or recombinantly produced. In one embodiment, the language “substantially free of cellular material” includes preparations of a polypeptide having less than about 30% (by dry weight) of a contaminating polypeptide, more preferably less than about 20% of a contaminating polypeptide, still more preferably less than about 10% of a contaminating polypeptide, and most preferably less than about 5% a contaminating polypeptide.

When the polypeptide or biologically active portion thereof is recombinantly produced, it is also preferably substantially free of culture medium, i.e., culture medium represents less than about 20%, more preferably less than about 10%, and most preferably less than about 5% of the volume of the polypeptide preparation.

The language “substantially free of chemical precursors or other chemicals” includes preparations in which the polypeptide is separated from chemical precursors or other chemicals that are involved in the synthesis of the polypeptide.

The present invention also provides antibodies that specifically bind to a polypeptide, or a portion thereof, as encoded by a nucleic acid described herein.

Antibodies can be made by many well-known methods (See, e.g., Harlow and Lane, “Antibodies; A Laboratory Manual,” Cold Spring Harbor Laboratory, Cold Spring Harbor, N.Y., (1988)). Briefly, purified antigen can be injected into an animal in an amount and in intervals sufficient to elicit an immune response. Antibodies can either be purified directly, or spleen cells can be obtained from the animal. The cells can then fused with an immortal cell line and screened for antibody secretion. The antibodies can be used to screen nucleic acid clone libraries for cells secreting the antigen. Those positive clones can then be sequenced. (See, for example, Kelly et al., 1992, Bio/Technology 10:163-167; Bebbington et al., 1992, Bio/Technology 10:169-175).

The phrases “selectively binds” and “specifically binds” with the polypeptide refer to a binding reaction that is determinative of the presence of the polypeptide in a heterogeneous population of polypeptides and other biologics. Thus, under designated immunoassay conditions, the specified antibodies bound to a particular polypeptide do not bind in a significant amount to other polypeptides present in the sample. Selective binding of an antibody under such conditions may require an antibody that is selected for its specificity for a particular polypeptide. A variety of immunoassay formats may be used to select antibodies that selectively bind with a particular polypeptide. For example, solid-phase ELISA immunoassays are routinely used to select antibodies selectively immunoreactive with a polypeptide. See Harlow and Lane, “Antibodies, A Laboratory Manual” Cold Spring Harbor Publications, New York, (1988), for a description of immunoassay formats and conditions that could be used to determine selective binding.

In some instances, it is desirable to prepare monoclonal antibodies from various hosts. A description of techniques for preparing such monoclonal antibodies may be found in Stites et al., eds., “Basic and Clinical Immunology,” (Lange Medical Publications, Los Altos, Calif., Fourth Edition) and references cited therein, and in Harlow and Lane “Antibodies, A Laboratory Manual” Cold Spring Harbor Publications, New York, 1988.

Throughout this application, various publications are referenced. The disclosures of all of these publications and those references cited within those publications in their entireties are hereby incorporated by reference into this application in order to more fully describe the state of the art to which this invention pertains.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

### Methods

For the purposes of the following examples, cell cultures utilized a well-characterized mouse preosteoblast cell line, MC3T3-E1 (ATCC, Manassas, Va.), which has been successfully used before and has a high osteoblastic potential (Zayzafoon et al., (2005) J Biol Chem 280:7049-7059; Shaulian & Karin (2001) Oncogene 20:2390-2400). Cells were maintained in Minimum Essential Medium Eagle, Alpha Modification (α-MEM) (Sigma) containing 10% fetal bovine serum (Atlanta Biologicals), 100 units/ml penicillin G and 100 μg/ml streptomycin (Invitrogen) at 37° C. with 5% CO2. Cells were passaged every 2-3 days and were not used beyond passage 25. Osteoblastic induction was performed by supplementing the maintenance medium with 5 mM P-glycerophosphate and 50 μM ascorbic acid-2-phosphate (Zayzafoon et al., (2005) J Biol Chem 280:7049-7059; Zayzafoon et al., (2002) J Biol Chem 277:37212-37218).

Primary osteoblasts were isolated from mouse calvariae. Mouse calvariae were dissected aseptically from one-day postnatal Balb/c mice. Frontal and parietal bones were cleaned of loose soft connective tissue and submerged in α-MEM. Calvariae were digested at 37° C. for 10 minutes with shaking in an enzymatic solution containing 0.1% collagenase and 0.05% trypsin containing 0.52 mM EDTA in α-MEM. This procedure was repeated to yield a total of five digests. The cells were collected by centrifugation at 1200 rpm for five minutes and were then resuspended in a-MEM containing 10% fetal bovine serum (FBS) and antibiotics (penicillin, 100 unit/mil and streptomycin, 100 mg/ml). Cells were grown to 70-80% confluence over the next three to four days at 37° C. in 5% carbon dioxide with humidification.

The polymerase chain reaction procedure involved the extraction of total RNA using the TRIzol method, as recommended by the manufacturer (Invitrogen). The yield and purity of RNA was estimated spectrophotometrically using the A260/A280 ratio. The quality of the RNA was examined by gel electrophoresis. One microgram of RNA was reverse transcribed using M-MLV reverse transcriptase and the equivalent of 10 ng will be used for the PCR reactions. Reverse transcriptions were carried out in a final volume of 25 μl containing 0.2 mM dNTPs, 120 nM of each primer and 1 U taq-DNA-polymerase. TaqMan real-time quantitative RT-PCR analysis was performed using primers for several genes that were previously used along with primers for 18S, which was used to normalize gene expression (TaqMan PCR detector 5700; Perkin-Elmer Applied Biosystems). PCR-amplified products were analyzed on 2% agarose gels containing ethidium bromide. The sequences and conditions for the specific primers used in this study were as previously described: ALP and osteocalcin (Zayzafoon et al., (2004) Endocrinology 145:2421-2432); c-fos (Miyamoto et al., (2001) Blood 98:2544-2554); fra-2 (Selvamurugan et al., (2004) J Biol Chem 279:27764-27773); NFATc1 and NFATc2 (Wilkins et al., (2002) Mol Cell Biol 22:7603-7613).

For whole cell protein extraction, cells were washed with chilled phosphate-buffered saline and centrifuged at 800 g for five minutes at 4° C., and resuspended in lysis buffer. A mixture of protease and phosphatase inhibitors (Zayzafoon et al., (2004) J Biol Chem 279:3662-3670) were added to the lysis buffer. Samples were then centrifuged at 14,000 rpm for thirty minutes at 4° C. and the supernatant protein concentration were measured using the Bio-Rad DC protein detection system (Zayzafoon et al., (2004) J Biol Chem 279:3662-3670).

For Western blots, whole cell extracts were loaded (30 μg/lane) on a 7.5% SDS mini-PAGE system. Following electrophoreses, proteins were transferred to polyvinylidene difluoride membrane, Immobilon-P (Millipore Co.), using a Bio-Rad wet transfer system. Protein transfer efficiency and size determination were verified using prestained protein markers. Membranes were blocked with Blotto B (Santa Cruz Biotechnology) for one hour at room temperature and subsequently incubated with antibodies directed against NFATc1, c2 and c3, Fra-2, SP-1 and actin (Santa Cruz Biotechnology). Signals were detected using a horseradish peroxidase-conjugated secondary antibody and an enhanced chemiluminescence detection kit (ECL, Amersham Bioscience).

Immunohistochemistry was performed in the following manner: MC3T3-E1 cells were cultured on cover slips for 24 hours. Cells were treated with CsA for 24 hours or with DMSO as a control. Cells were then washed with PBS, fixed for 5 minutes with chilled methanol and permeabilized by 0.1% Triton X-100 in PBS for 5 min. Endogenous peroxidase activity was quenched using 1% hydrogen peroxide. Samples were blocked for one hour in Fc Receptor Blocker (Innovex Biosciences, Richmond, Calif.). Primary antibodies were diluted in Fc Blocker solution and applied to the sections overnight at 4° C. FITC-conjugated secondary antibodies were then used for thirty minutes. Samples were incubated in peroxidase substrate for 30 seconds, dehydrated, mounted and cover-slipped. Negative controls were processed alongside the examined tissue, but rabbit IgG was used instead of the primary antibody. At least ten randomly selected microscopic fields were examined using a 10× and 40× objective. Photos were taken using a SPOT digital camera. These studies were previously performed successfully on cells cultured on cover slips and bone sections obtained from paraffin embedded tissue.

Alkaline phosphatase staining for the following examples was performed as follows. Osteoblasts were fixed in 2% PAF/PBS for ten minutes and stored at 4° C. in 0.1 M cacodylate buffer, pH 7.4. Cells were then incubated at 37° C. with freshly prepared alkaline phosphatase substrate solution (100 mM Tris-Maleate buffer (pH 8.4), 2.8% N,N-dimethyl formamide (v/v), 1 mg/ml Fast Red TR and 0.5 mg/ml naphthol AS-MX phosphate). The reaction was terminated after 30 minutes by removal of the substrate solution and washing with 100 mM cacodylic acid buffer (Zayzafoon et al., (2005) J Biol Chem 280:7049-7059). Mineralization was assessed by either von Kossa staining of the cultures (5 minutes in 3% AgNO3) (Zayzafoon et al., (2005) J Biol Chem 280:7049-7059) or by measurement of calcium deposition as previously described (Zayzafoon et al., (2004) J Biol Chem 279:3662-3670). Briefly, cells were collected by scraping into 10 mM TBS (pH 7.2) containing 0.2% Triton X-100. An aliquot was removed for determination of protein concentration and the remaining material was incubated in 0.5 M HCl (final concentration) at 70° C. for 15 minutes, followed by spectrophotometric calcium detection (Arsenazo III, Sigma-Aldrich Corp, St. Louis, Mo.).

In addition, alkaline phosphatase activity was quantified by incubating osteoblast homogenates with p-nitrophenyl phosphate for thirty minutes at 37° C., absorbance was read at 410 nm.

For electrophoretic mobility shift assays (EMSA), nuclear extracts were incubated for twenty minutes at room temperature with ³²P-labeled oligonucleotides containing the appropriate consensus sequences. The AP-1 oligonucleotide was labeled as previously described (Zayzafoon et al., (2005) J Biol Chem 280:7049-7059; Zayzafoon et al., (2004) J Biol Chem 279:3662-3670). The oligonucleotide sequence used as a probe was as follows: 5′ -GCGTTGATGAGTCAGCCGGAA-3′ (SEQ ID NO:1; Sunters et al., (2004) J Biol Chem 279:9882-9891). Nuclear extracts were prepared by washing MC3T3-E1 cells with chilled PBS and centrifuging at 800 g for 5 minutes at 4° C. Nuclei were then isolated by detergent lysis of the cells with an NP40 lysis buffer (Zayzafoon et al., (2005) J Biol Chem 280:7049-7059). Nuclei were then treated with a hypotonic solution followed by a 30-minute incubation at 4° C. in extraction buffer (Zayzafoon et al., (2005) J Biol Chem 280:7049-7059). Nuclei were finally centrifuged at 14,000 G for 30 minutes at 4° C. and the supernatant protein concentration was measured by the Bio-Rad DC Protein Assay. All solutions in this procedure contained a cocktail of protease and phosphatase inhibitors (Zayzafoon et al., (2004) J Biol Chem 279:3662-3670). Four μg of nuclear extracts were then incubated for 20 minutes at room temperature with a ³²P-labeled oligonucleotide containing the AP-1 consensus sequence in loading buffer and 50 μg/ml poly (dI-dC) (Zayzafoon et al., (2005) J Biol Chem 280:7049-7059).

DNA-protein complexes were resolved on 5% native polyacrylamide gels. Gels were dried and exposed to X-ray film, at 80° C., with an intensifying screen.

Transient transfections was performed as described herein. Cells were plated at a density of 2×10⁴ cells/cm² in a 6-well plate. Twenty-four hours after plating, cells were transfected with 1 μg of various luciferase reported plasmids including those driven by the AP-1, TCF/LEF (Invitrogen), Runx2-Luc, or CMV-β galactosidase reporter construct (as a control). Transfection was performed using LipofectAMINE (Invitrogen) according to the manufacturer's instructions. Twenty-four hours post-transfection, the medium was changed and cells were treated with calcineurin inhibitors as indicated. Cells were then lysed and reported activity read suing a luciferase (Promega) or galactosidase (CLONTECH) assay system and a luminometer. Transfection efficiency was evaluated by transfecting CMV-GFP and an efficiency of 75-90% has been observed.

Gene silencing was performed in accordance with the following method. MC3T3-E1 cells were plated at 50-70% confluence or at a density of approximately 2×10⁴ cells/cm² in a 6-well plate. Calcineurin, NFATc1, or control non-functional siRNA (Santa Cruz Biotechnology) were transfected into cells using TransIT-TKO transfection reagent as recommended by the manufacturer, Mirus (Madison, Wis.). Medium was changed 24 hours post transfection and the cells were cultured with fresh medium. Cells were harvested 48 hours later. The success of the siRNA was confirmed by demonstrating the inhibition of protein expression.

For animal studies, 5 day old Balb/c mice were injected daily in the calvarial region with 100 μl PBS solution containing either DMSO (control) or 1 μM CsA as previously described (Zayzafoon et al., (2005) J Biol Chem 280:7049-7059). Briefly, a needle (26 gauge) was inserted at the base of the calvaria and pushed until it reached the central region of the skull. Solution was then injected subcutaneously over the parietal region of the skull. After 10 days, mice were sacrificed and calvariae were harvested. Tissues were washed with PBS, fixed with 10% formalin and embedded in methyl methacrylate. All animal studies were approved by the Institutional Animal Care and Use Committee of the University of Alabama at Birmingham.

For some of the following examples, histology and histomorphology was performed by cutting longitudinal undemineralized sections (5 μm thick) cut from Methyl Methacrylate (MMA) plastic embedded blocks and stained with Goldner's Trichrome for the static measurements or left unstained for dynamic (fluorescent) measurements.

A region of interest was selected and remained constant for all animals regardless of the shape of the section. Standard bone histomorphometry as described by Parfitt et al. (Parfitt et al., (1987) J Bone Miner Res 2:595-610) was performed using the Bioquant Image Analysis software (R & M Biometrics, Nashville, Tenn.) Four types of primary measurements were made - area, length (or perimeter), distance between points or lines, and numbers. These referents, such as tissue volume, bone volume, bone surface, and osteoid surface were used to derive other indices, such as trabecular number and trabecular separation.

Dynamic measurements were performed in the same region of interest using the unstained section. Fluorescent measurements were made of single-labeled surface, double-labeled surface, and interlabel width. By applying the interlabel period, it was possible for the software to calculate the Mineral Apposition Rate, as well as formation and resorption rates, and remodeling cycle duration.

All statistical analyses were performed using the Microsoft Excel data analysis program for Student's t-test analysis. Experiments were repeated at least three times unless otherwise stated. Values were expressed as a mean ±SE.

### Example 1

### Expression of NFAT Isoforms

MC3T3 cells were cultured and treated with 1 μM CsA for eleven days. Cytoplasmic and nuclear proteins were extracted. The extracts were placed into separate lanes of an SDS-PAGE gel in the amount of 20 μg per lane. Separation was run with SDS-PAGE followed by immunoblots developed using antibodies directed against total NFATc1, c2, and c3.

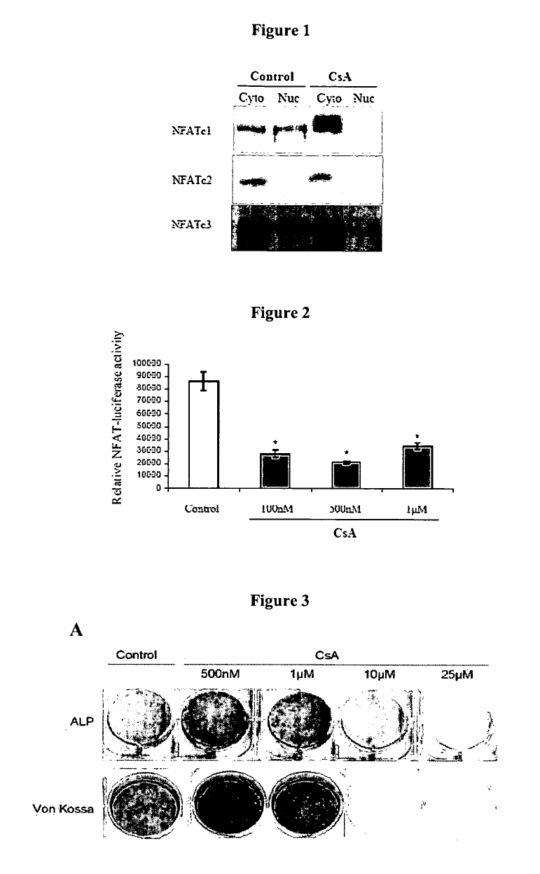

This analysis demonstrated that differentiated osteoblasts express NFATc1, c2, and c3. NFATc1 and c3 were detected in both the cytoplasm and the nucleus of osteoblasts while NFATc2 was only detectable within the cytoplasm. The osteoblasts treated with CsA exhibited markedly decreased amounts of NFATc1 and c3 in the nucleus, indicating a definite decrease in the amount of translocation of NFATc1 and c3 into the nucleus. FIG. 1 shows representative images of three separate immunoblots demonstrating an increased amount of NFATc1 and c3 in the cytoplasm as compared to the nucleus of the MC3T3 cells after exposure to CsA over the control, which shows amounts of the c1 and c3 isomers in the nucleus. Positive controls, proteins extracted from Jurkat cells, were examined and show expression of NFATc1, c2 and c3 (data not shown). NFAT protein expression is also confirmed by demonstrating that osteoblasts express the genes for NFATc1, c2 and c3 as shown by RT-PCR. NFAT gene expression was not changed after CsA treatment (data not shown).

Real-time and regular RT-PCR are performed to examine the RNA levels of the NFAT isoforms and confirm the protein results.

### Example 2

### Decrease In NFAT Transactivation

MC3T3 cells were cultured for 24 hours and then transfected with a NFAT-luciferase construct (BD Biosciences). Briefly, the pNFAT-TA-luciferase construct contains three tandem copies of the NFAT consensus sequence (http://www.clontech.com/clontech/techinfo/vectors/vectorsm-q/pdf/pt3369-5w.pdf). 24 hours after transfection, cells were treated with 100 nM, 500 nM or 1 μM CsA for an additional 24 hours. Cells were then harvested, lysed, and luciferase activity was measured. The amount of CsA treatment that the transfected cells received varied in order to ascertain concentration effects of the CsA on NFAT activity.

This analysis demonstrated that CsA inhibits the transactivation of NFAT at concentrations as low as 100 nM and remains inhibited at 1 μM. Accordingly, the ability of CsA to inhibit NFAT activity within osteoblasts is dose dependent. FIG. 2 is a graph of the results of this study. NFAT-luciferase activity was plotted against a control, not exposed to CsA, and three concentrations—100 nM, 500 nM, and 1 μM of CsA. It is clear that exposure of the cells to CsA decreased the NFAT activity within those cells.

### Example 3

### Increased Osteoblast Differentiation In Vitro

In order to directly determine the role of NFAT in osteoblast differentiation, MC3T3 pre-osteoblasts were cultured for 14 days or 21 days in the presence of β-glycerophosphate and ascorbic acid. Cells were treated with different concentrations of CsA or FK506 every 2 days, starting on day 4 and for the duration of the culture. A control cell culture was treated with DMSO alone. After the term of the study, the cells were fixed and stained for alkaline phosphatase activity and for mineralization by von Kossa.

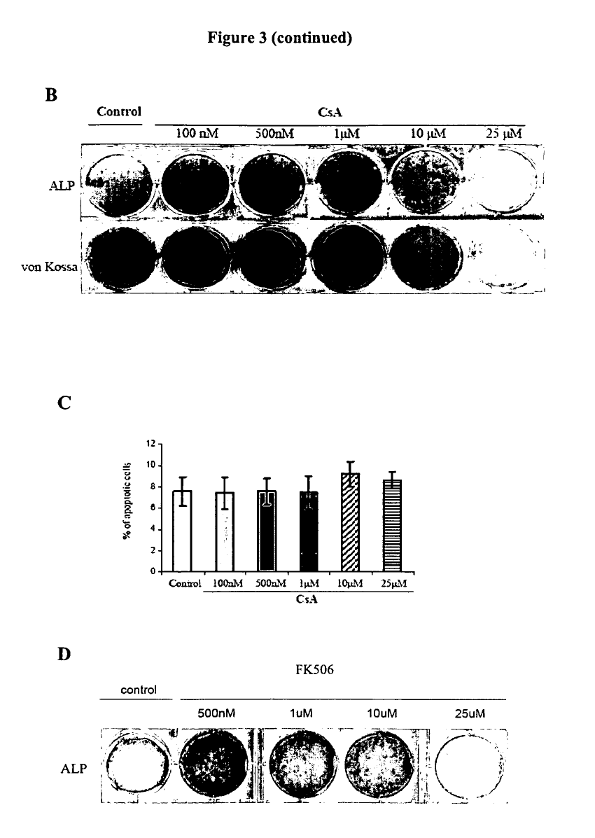

This analysis demonstrated that both alkaline phosphatase and mineralization were increased dose dependently in response to CsA treatment. This increase reached a plateau when osteoblasts were treated with 500 nM CsA. This increase in both alkaline phosphatase and mineralization indicates that NFAT negatively regulates osteoblast differentiation and that inhibition of NFAT by CsA, in turn, increases osteoblast differentiation. FIG. 3A is a photograph of the MC3T3 cell cultures contacted with no CsA, 500 nM, 1 μM, 10 μM, or with 25 μM CsA. The images on the top measure alkaline phosphatase activity while the images on the bottom measure mineralization - both are indications of osteoblast cell differentiation. Similar results were seen in three separate experiments. FIG. 3B is a photograph of the primary mouse calvarial osteoblast cell cultures contacted with no CsA, 100 nM, 500 nM, 1 μM, 10 μM, or with 25 μM CsA. Similar results were seen in three separate experiments.

Similarly, alkaline phosphatase and mineralization increased dose-dependently in response to FK506 treatment, where cell cultures were contacted with no FK506, or with 500 nM, 1 μM, 10 μM, or 25 μM FK506 (FIG. 3d). This increase reached a plateau when osteoblasts were treated with 500 nM and 1 μM FK506. This increase in both alkaline phosphatase (FIG. 3d) and mineralization (data not shown) indicates that NFAT negatively regulates osteoblast differentiation and that inhibition of NFAT by FK506, in turn, increases osteoblast differentiation.

In addition, MC3T3-E1 cells were cultured for 4 days and treated with different doses of CsA for 24 hours. Cells were then harvested and washed with PBS. Cells were stained with fluorescein isothiocyanate-conjugated annexin V and PI, and apoptotic cell death was analyzed by flow cytometry. The values shown in the graph in FIG. 3c represent the mean ±SE of 4 separate experiments each performed in triplicate (*, p<0.05 compared to control). The results demonstrate that the treatment of the cells with CsA at concentrations which have an effect on alkaline phosphatase activity and mineralization, did not have a significant effect on the rate of apoptotic cell death of cells in the culture.

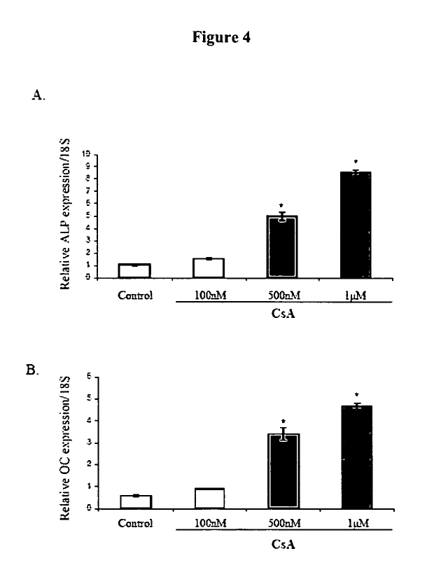

RT-PCR analysis, shown in FIGS. 4a and 4b, demonstrate CsA dose dependently increased the expression of markers known to identify osteoblast differentiation. FIG. 4a shows the increase in alkaline phosphatase expression, an early osteoblastic gene marker, from the control through 100 nM, 500 nM, and 1 μM of CsA exposure. Likewise, FIG. 4b shows the increase in osteocalcin expression, a late osteoblastic gene marker, from the control through 100 nM, 500 nM, and 1 μM of CsA exposure.

### Example 4

### Increased Osteoblast Differentiation In Vivo

DMSO, as control, or 1 μM CsA were dissolved in PBS and injected subcutaneously for 10 days into the calvarial region of 5-day-old mice. At the termination of the study the mice were sacrificed, calvariae dissected, and processed for staining and bone histomorphometry. Staining of the calvariae with modified Goldner's TriChrome and von Kossa staining was performed.

This study demonstrated that treatment with CsA dramatically increased the number of osteoblasts present, as well as increasing newly formed bone, bone volume, and bone mineralization. Histomorphometric analysis of calvariae demonstrated a 31% increase in osteoblast number and an 18% increase in bone volume. These results further demonstrated CsA's ability to increase osteoblast formation and differentiation in vivo. FIGS. 5a and 5b demonstrate the increase in both the number of osteoblasts present after treatment (5a) as well as in increase in bone volume (5b). FIG. 5a shows the 31% increase in osteoblasts after exposure to 1 μM CsA. FIG. 5b shows the 18% increase in bone volume as compared to total volume.

These experiments are repeated with primary osteoblast cell lines and confirm that CsA increases the number of osteoblasts formed as well as increases bone mineralization.

### Example 5

### Increased Fra-2 Expression In Osteoblasts

MC3T3 cells were cultured for eleven days and treated with CsA whereas control cells were treated with DMSO alone. At the end of the study cells were harvested for RNA and nuclear protein extraction. Real time RT-PCR was performed for fra-2, c-jun, JunB and c-fos. Values were obtained from three experiments and represent the mean plus or minus S.E. of fra-2 mRNA expression relative to 18S rRNA expression.

Immunoblots of the nuclear extracts were developed using antibodies directed against Fra-2 and total SP-1. MC3T3 cells were cultured for 2 days on coverslips. Cells were treated with CsA (1 μM) for 24 hours. Cells were then fixed and immunohistochemistry for Fra-2 was performed.

CsA dose-dependently decreased expression of c-fos, while fra-2 expression and nuclear translocation increased within a cell when exposed to CsA. Moreover, the increase in fra-2 expression was dependent upon the dose of CsA administered. Immunohistochemistry demonstrated that CsA increases Fra-2 nuclear protein expression after twenty-four hours of treatment. It was further demonstrated that this increase in Fra-2 expression resulting from treatment with CsA caused an increase in the activity of AP-1 DNA binding. Treatment with CsA dose-dependently decreased the expression of c-fos and increased the expression of fra-2 in osteoblasts. There was no significant change in the gene expression of c-jun and JunB.

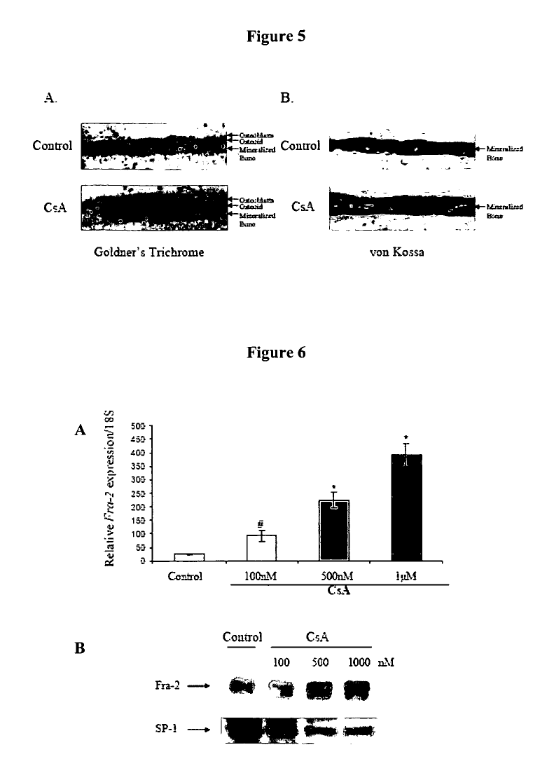

FIG. 6a shows the CsA dose dependent increase in Fra-2 expression. Specifically, the figure shows the relative fra-2 expression compared to 18S expression as the CsA concentration was increased from zero, control, through 100 nM, 500 nM, and 1 μM. FIG. 6b shows the results of an immunoblot using antibodies directed against Fra-2 and total Sp-1 (control). As the concentration of CsA was increased from zero, control, through 100 nM, 500 nM, and 1 μM, it was apparent that the amount of Fra-2 increased as compared to Sp-1. FIG. 6c shows the result of performing immunohistochemistry for Fra-2, demonstrating an increase in Fra-2 nuclear protein expression in response to CsA dosing.

Fra-2 is a member of the Fos family of AP-1 transcription factors that form heterodimers with the AP-1 Jun family members (c-jun, JunB and JunD) and functions as a transcriptional regulator (Suzuki et al., (1991) Nucleic Acids Res., 19:5537-5542). Therefore, the effect of CsA on AP-1 DNA binding activity was examined by EMSA. MC3T3-E1 pre-osteoblastic cells were cultured for 11 days and treated with different concentrations of CsA. Nuclear proteins were extracted and incubated with radiolabeled oligonucleotide containing an AP-1 consensus sequence and an EMSA was performed. Inhibition of NFAT by CsA increased AP-1 DNA binding activity dose-dependently in osteoblasts (data not shown). Super shift analyses using antibodies to AP-1 family members confirmed that Fra-2 and JunD were increased in the AP-1 DNA-protein complex. However, Fra-1, c-Fos and junB AP-1 DNA binding was not affected by inhibition of NFAT (data not shown). These findings suggest that inhibition of NFAT increases osteoblast differentiation by increasing Fra-2 expression, leading to an increase in Fra-2/JunD binding to the AP-1 consensus sequence.

### Example 6

### The C1 Isoform of NFAT Negatively Regulates Fra-2 Expression

MC3T3 cells were cultured and transfected with control siRNA and NFATc1 siRNA 24 hours after transfection. Approximately 72 hours after transfection, cells were harvested and lysed for whole cell extraction. Immunoblots were developed using antibodies directed against either NFATc1 or Fra-2.

This study demonstrated that after siRNA transfection, NFAT_{c1} protein expression was inhibited in comparison to cells transfected with a non-functional scrambled siRNA. However, the inhibition of NFATc1 expression induced Fra-2 expression, in comparison with control siRNA treatment.

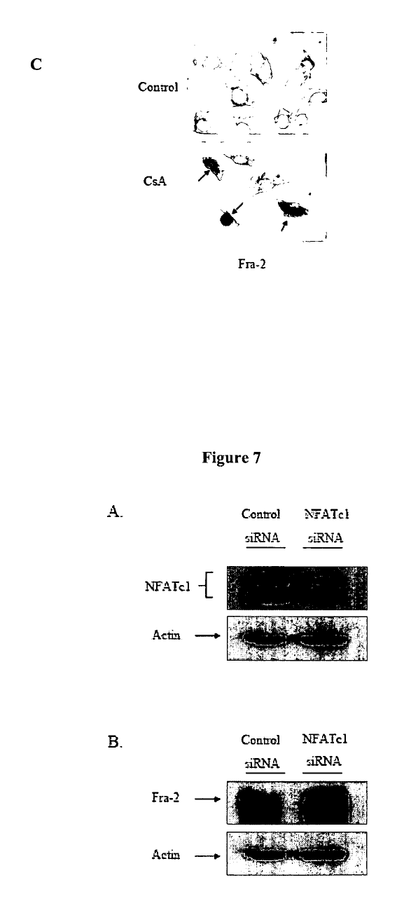

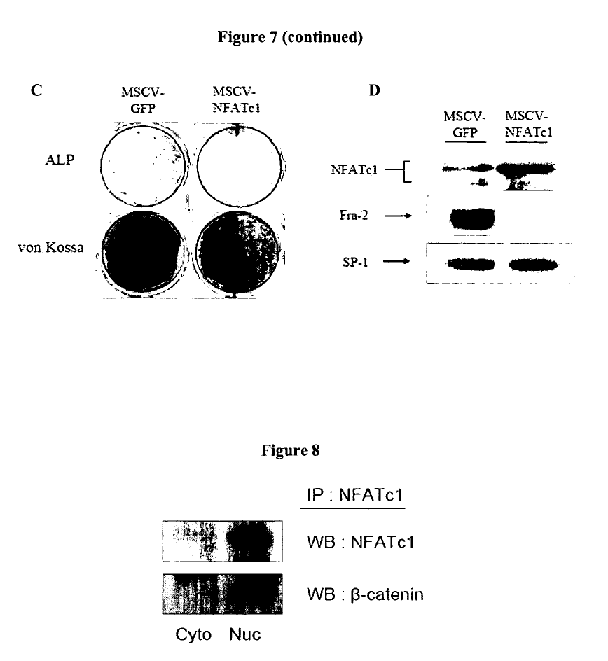

FIG. 7a shows immunoblots against NFATc1 and FIG. 7b shows immunoblots against Fra-2.

SiRNA for the remaining NFAT isoforms and for CnA and CnB is administered to MC3T3 cells and to primary osteoblast cell lines to determine whether the results are specific for NFATc1 and NFATc3, or whether the remaining isoforms also play a role.

Furthermore, constitutively-active NFATc1 was expressed in MC3T3-E1 cells using a retroviral overexpression system (Dr. Neil Clipstone). Transduction efficiency was approximately 95%. Over-expression of constitutively-active NFATc1 in MC3T3-E1 osteoblasts inhibited osteoblast differentiation as demonstrated by a decrease in ALP activity and mineralization (FIG. 7c). In addition, over-expression of the constitutively active NFATc1 dramatically decreased Fra-2 expression in comparison with control MSCV-GFP infected cells (FIG. 7d).

### Example 7

### The C1 Isoform of NFAT Interacts With β-Catenin

MC3T3 cells were cultured for four days without osteogenic induction. This time point was selected as the cells are considered pre-osteoblasts and are unable to express osteoblast gene markers. Cytoplasmic and nuclear proteins were extracted and immunoprecipitation was performed using agarose beads conjugated with NFATc1 antibody. Western blots were developed using antibodies directed against NFATc1 to confirm the success of the immunoprecipitation and for β-catenin to confirm the physical interaction.

This study demonstrated that NFATc1 binds to β-catenin. FIG. 8 shows the results of immunoblots were developed using antibodies directed against NFATc1 (upper) or β-catenin (lower).

### Example 8

### Calcineurin Conditional Knockout In Osteoblast Specific Cn^{-/-} Mouse

A conditional knockout of calcineurin in osteoblasts was made (Cn-b1). Mice expressing CnB-LoxP were crossed with mice expressing Cre recombinase under the control of the osteocalcin gene.

The extent of calcineurin gene and protein expression in different tissues, including bone, collected from wild type and knockout animals is determined by RT-PCR and Western blot analyses. In addition, the activation of NFAT is determined by immunofluorescence using specific antibodies to detect nuclear localization of the NFAT isoforms. Lean and fat body mass evaluation, radiographs of the skeleton, knees and hind limbs, is performed. Bone mineral density and bone mineral content are also evaluated.

Blood samples are taken from animals at the time of sacrifice to examine markers of bone turnover: BSAP and osteocalcin for formation, and NTX for resorption. Bone is harvested and histological analyses of the calvariae, tibial metaphyses, periosteum and edosteum of the femoral diaphsis are performed on frozen, paraffin or plastic embedded tissue for H&E and Trichrome staining. This allows for the determination of the histomorphometric parameters of bone, most importantly, bone thickness, osteoid surface and osteoblast and osteoclast number using BioQuant image analysis.

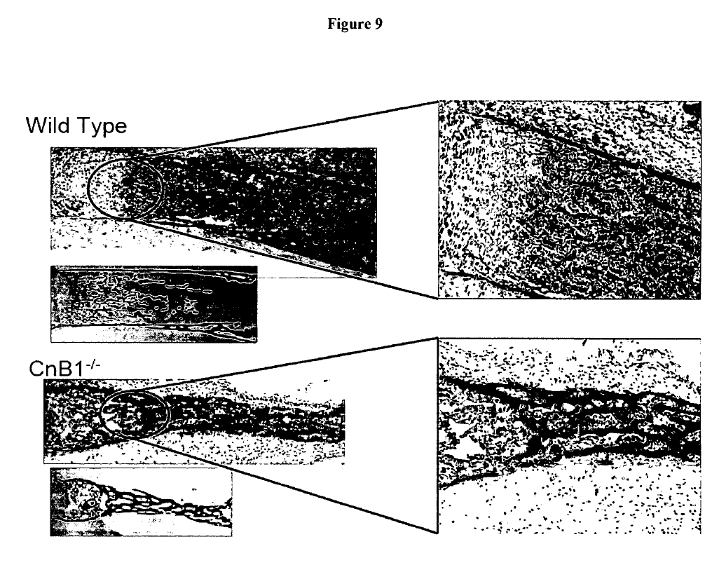

Preliminary results (n=2) demonstrate that osteoblast-specific Cn-b1 conditional knockout mice exhibit an osteopetrotic phenotype driven by a dramatic increase in bone formation and mineralization (FIG. 9). Histomorphometrically, Cn-b1 osteoblast specific knockout mice, compared to wild type, have increase in bone volume/total volume (260%) and osteoblasts numbers/mm (500%) with no change in osteoclasts numbers/mm.

This newly generated mouse develops osteopetrosis due to significant and massive bone formation. This supports the finding that NFAT negatively regulates osteoblast differentiation by demonstrating that in the absence of the NFAT pathway inhibitor, osteoblasts proliferate and produced too much bone. These studies suggest that the specific inhibition of calcineurin by low concentrations of CsA may represent a viable anabolic therapy for osteopenic conditions.

### Example 9

### Osteoblasts Derived Cn^{-/-} Mouse Display Enhanced Differentiation Patterns

Osteoblast precursors are isolated from wild type and Cn^{-/-} mice. Cells are cultured for 4, 14, and 21 days. At the end of the study, cultures are stained for alkaline phosphatase and von Kossa. Also, RNA is extracted and regular and real-time RT-PCR is performed examining the gene expression of osteoblast markers such as alkaline phosphatase, osteocalcin, and collagen I.

The differentiation of osteoblasts derived from Cn^{-/-} mice is greater than from wild type. These cells exhibit increased alkaline phosphatase and von Kossa staining. Furthermore, the osteoblast gene expression is increased in cells derived from the mutant mice. Finally, the basal expression of Fra-2 and Runx2 in the mutant osteoblasts is higher than in wild type osteoblasts.

1. A method of identifying a compound capable of increasing osteoblast cell differentiation, comprising:
a) providing a bone precursor cell,
b) contacting the bone precursor cell with a test compound, and
c) determining whether a decrease in nuclear translocation of a member of the nuclear factor of activated T-cells (NFAT) superfamily occurs in the cell contacted with the compound, said decrease being an indication that the compound increases osteoblast cell differentiation.

2. (canceled)

3. The method of claim 1, wherein the member of the NFAT superfamily is selected from the group consisting NFATc1 and NFATc3.

4. The method of claim 3, wherein the member of the NFAT superfamily is NFATc1.

5. The method of claim 1, wherein the bone precursor cell expresses calcineurin, and wherein the test compound decreases the binding of calcineurin to a member of the NFAT superfamily.

6. (canceled)

7. A method of identifying a compound capable of increasing osteoblast cell differentiation, comprising:
a) providing a bone precursor cell expressing a member of the NEAT superfamily,
b) contacting the bone precursor cell with a test compound, and
c) determining whether an increase of Runx-2 expression occurs in the cell contacted with the compound, said increase being an indication that the compound increases osteoblast cell differentiation.

8. The method of claim 7, wherein contact with the test compound results in a decrease in dephosphorylation of the member of the NFAT superfamily.

9. (canceled)

10. The method of claim 8, wherein the member of the NFAT superfamily is selected from the group consisting NFATc1 and NFATc3.

11. The method of claim 10, wherein the member of the NFAT superfamily is NFATc1.

12. The method of claim 7, wherein the cell expresses calcineurin, and wherein the test compound decreases the binding of calcineurin to a member of the NFAT superfamily.

13. The method of claim 7, further comprising the step of:
d) manufacturing the compound.

14. (canceled)

15. (canceled)

16. (canceled)

17. (canceled)

18. (canceled)

19. (canceled)

20. (canceled)

21. (canceled)

22. (canceled)

23. (canceled)

24. The method of claim 1, further comprising the step of:
d) manufacturing the compound.

25. A method of differentiating a bone precursor cell population comprising:
a) providing a bone precursor cell population, and
b) contacting the bone precursor cell population with an effective amount of an inactivator of the calcineurin signaling pathway
wherein differentiation of the bone precursor cell population to osteoblasts is increased in comparison to a bone precursor cell population that is not contacted with an effective dose of the inactivator of the calcineurin signaling pathway.

26. The method of claim 25, wherein the bone precursor cell population is contacted with the inactivator for greater than approximately 24 hours.

27. The method of claim 25, wherein the inactivator is cyclosporine or FK506.

28. The method of claim 27, wherein the inactivator is cyclosporine is present at a concentration of approximately 1 nM to approximately 1 μM.

29. (canceled)

30. The method of claim 27, wherein the inactivator is FK506 present at a concentration of approximately 500 nM to approximately 25 μM.

31. The method of claim 25, wherein greater than approximately 25% of the differentiated cell population comprises osteoblasts.

32. A composition comprising a homogenous population of differentiated cells, wherein the differentiated cells are differentiated from bone precursor cells in an in vitro culture, and wherein greater than approximately 25% of the population express Runx2, Fra-2, and alkaline phosphatase.

33. The composition of claim 32, wherein the cells have been differentiated by contact with an effective amount of an inactivator of the calcineurin signaling pathway.

34. The composition of claim 33, wherein the inactivator is cyclosporine or FK506.

35. The composition of claim 34, wherein the inactivator is cyclosporine present at a concentration of approximately 1 nM to approximately 1 μM.

36. (canceled)

37. The composition of claim 34, wherein the inactivator is FK506 present at a concentration of approximately 500 nM to approximately 25 μM.

38. The composition of claim 32, wherein greater than approximately 25% of the population express one or more of the markers selected from the group consisting of osteocalcin, P-catenin, CCAAT/enhancer binding protein (c/EBP), and ATF4.

39. The composition of claim 32, wherein greater than approximately 50% of the population are osteoblasts.

40. The composition of claim 32, wherein greater than approximately 60% of the population are osteoblasts.

41. The composition of claim 32, wherein greater than approximately 70% of the population are osteoblasts.

42. A method of improving bone mass in an individual in need thereof comprising administering to the individual a therapeutically effective amount of a compound that decreases nuclear translocation or dephosphorylation of a member of the NFAT superfamily in a bone precursor cell.

43. (canceled)

44. The method of claim 43, wherein the member of the NFAT superfamily is selected from the group consisting NFATc1 and NFATc3.

45. The method of claim 44, wherein the member of the NEAT superfamily is NFATC1.

46. The method of claim 42, wherein the compound decreases the binding of calcineurin to a member of the NFAT superfamily.

47. The method of claim 42, wherein the compound is cyclosporine at a concentration from approximately 10 nM to approximately 0.5 μM.

48. (canceled)

49. The method of claim 42, wherein the compound is FK506 at a concentration from approximately 500 nM to approximately 25 μM.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. A pharmaceutical composition capable of improving bone mass, comprising a therapeutically effective amount of a compound that decreases nuclear translocation or dephosphorylation of a member of the nuclear factor of activated T-cells (NFAT) superfamily in a bone precursor cell, whereby administration of the composition causes an improvement in bone mass in vivo.

67. The composition of claim 66, wherein the member of the NFAT superfamily is selected from the group consisting NFATc1 and NFATc3.

68. The composition of claim 67, wherein the member of the NFAT superfamily is NFATc1.

69. The composition of claim 66, wherein the compound decreases the binding of calcineurin to a member of the NFAT superfamily.

70. (canceled)

71. (canceled)

72. (canceled)

73. (canceled)

74. (canceled)

75. (canceled)

76. (canceled)

77. (canceled)

78. (canceled)
